(19) United States
(12) Patent Application Publication
(10) Pub. No.: US 2007/0269877 A1
(43) Pub. Date: Nov. 22, 2007

(54) Dna polymerases

(75) Inventors: Bernard Connolly, Newcastle Upon Tyre (GB); Ben Biles, Eastleigh (GB)

Correspondence Address: PILLSBURY WINTHROP SHAW PITTMAN LLP, P.O BOX 10500, McLean, VA, 22102, US

(73) Assignee: The University of Newcastle, Newcastle Upon Tyne (GB)

(21) Appl. No.: 11/662,987

(22) PCT Filed: Aug. 26, 2005
(86) PCT No.: PCT/GB05/03341

§ 371 Date: Mar. 15, 2007

(30) Foreign Application Priority Data
Sep. 15, 2004 (GB) 0420500.1

(51) Int. Cl. C12N 9/00; C07H 21/00

(52) U.S. Cl. 435/183; 536/23.2; 536/25.3

(57) ABSTRACT

The present invention relates to a variant archaeal DNA polymerase comprising a modified amino acid sequence of a wild-type amino acid sequence, wherein during DNA replication, the variant polymerase is adapted to mis-incorporate a greater number of incorrect bases than compared to a wild-type archaeal DNA polymerase. Such a variant DNA polymerase may be usefully employed in biological assay systems, such as PCR.

The present invention relates to DNA polymerases, and particularly to DNA polymerase variants, and their use in random mutagenesis protocols, including error prone PCR.

The polymerase chain reaction (PCR) is a method whereby a sequence of DNA may be selectively amplified to produce a large sample that may be readily analysed. A solution containing the DNA to be amplified, together with free bases, a polymerase enzyme and primers that bind to opposite ends of the two strands of the DNA segment to be replicated, is heated to break the bonds between the strands of DNA. When the solution cools, the primers bind to the separated strands and the polymerase builds a new strand by joining free bases to the primers thereby producing a new strand that is restricted solely to the desired segment. PCR enables billions of copies of a small piece of DNA to be produced in several hours.

Heat stable polymerases are required for this DNA amplification process and one of the most commonly used is Taq DNA polymerase, isolated from the bacterium Therimus aquaticus, a member of the prokaryotic domain. Many archaeal DNA polymerases are also thermally stable and so have been extensively used in PCR, e.g. the enzyme isolated from Pyrococcus furiosus (Pfu DNA polymerase). Given the utility of archaeal DNA polymerases in PCR, the inventors of the application in suit decided to make modifications of the polymerase with a view to producing mutant archaeal DNA polymerases that are especially adapted for use in particular PCR protocols.

One such PCR protocol is the use of PCR to generate random mutations in DNA. Random mutagenesis is an important technique that may be used to randomly change DNA sequences, and hence alter amino acids of proteins expressed therefrom. Random mutagenesis is most commonly used to study protein structure-function relationships and for altering proteins to improve or vary their characteristics.

Error prone PCR is a random mutagenesis technique for generating amino acid substitutions in proteins by introducing mutations into a gene during PCR. Mutations are deliberately introduced using an error prone polymerase or conditions that favour mistakes. An error prone polymerase replicates from the nucleic acid with low fidelity and, in complete contrast to the normal “high fidelity” requirements of PCR, will be designed to result in error prone PCR. The mutated PCR products are cloned into an expression vector and the library can be screened for changes in protein properties. For example, the proteins may then be assayed and the effect of a mutation on function assessed. Hence, random mutagenesis allows the assignment of function to specific amino acids/regions or domains of proteins and the identification of beneficial properties when structural data is missing or when such mutations are difficult to predict even with a structure.

Like all PCR-based methods, error prone PCR requires a thermostable DNA polymerase. So far, error prone PCR has been carried out with Taq polymerase, either by using Mn²⁺ and unbalanced dNTP levels, or mutants of Taq. However, unfortunately, such methods are associated with poor yields, low levels of mutation and/or a biased mutation spectrum.

Hence, it is an aim of embodiments of the present invention to address the problems with the prior art, and to provide an error prone DNA polymerase, having a reduced fidelity during DNA replication, which may be used in improved methods of random mutagenesis.

Accordingly, a first aspect of the present invention provides a variant archaeal DNA polymerase comprising a modified amino acid sequence of a wild-type amino acid sequence, wherein during DNA replication, the variant polymerase is adapted to mis-incorporate a greater number of incorrect bases than compared to a wild-type archaeal DNA polymerase.

During DNA replication, DNA polymerase copies or replicates a double stranded template DNA into two daughter strands, which form a DNA molecule identical to the template DNA. Hence, the DNA replication process ensures that progeny accurately inherit genetic material from parents. Central to this process is (i) DNA base pairing (Adenine always pairing with Thymine, and Guanine always pairing with Cytosine), and (ii) the ability of a DNA polymerase to copy Adenine with Thymine, Thymine with Adenine, Guanine with Cytosine, and Cytosine with Guanine to maintain base pairing.

While the DNA replication system in most species has a high fidelity, errors do occur in which incorrect bases can be incorporated in to the daughter DNA strand thereby causing a mutation. Several types of mutation may occur: (i) the substitution of one base pair for another; (ii) the deletion of one or more bases; and (iii) the insertion of one or more bases. In order to ensure as high a fidelity as possible many DNA polymerases possess, in addition to the 5′-3′ polymerase activity, a 3′-5′ proof-reading exonuclease activity, which is required to remove bases that have been incorrectly incorporated in the daughter DNA. The archaeal DNA polymerases that are the subject of the present invention comprise a 3′-5′ proof-reading exonuclease.

The present invention is based upon research (see the Examples) conducted by the inventors that has identified a number of mutant archaeal DNA polymerase enzymes that are adapted to (intentionally) mis-incorporate bases into the daughter strand during DNA replication at a rate higher than the wild-type polymerase from which they are derived. They realised that these variant polymerases according to the invention may be usefully employed in random mutagenesis protocols as described herein.

Hence, the inventors have now produced a DNA polymerase in accordance with the present invention that shows reduced accuracy or fidelity while replicating DNA. For example, Adenine is not only correctly copied with Thymine, but a significant degree of mis-incorporation (insertion of bases other than Adenine) also takes place. The same applies for Thymine, Guanine and Cytosine. As a result, the DNA produced by the variant DNA polymerase (the daughter strand) is not a faithful correct copy of the parental DNA (the template), but contains a significant number of randomly introduced errors, i.e. changes in base sequence.

Hence, by the term “mis-incorporate incorrect bases”, we mean the variant DNA polymerase, during replication of the parental DNA, (i) may incorporate into the daughter strand, a base other than that which would normally be incorporated to conform with traditional base pairing, i.e. a base substitution; (ii) may delete one or more bases; and (iii) may insert one or more bases. This is also known as low fidelity replication and results in the production of mutations in the daughter DNA.

It is preferred that the in vitro rate of mutation using the variant polymerase according to the invention (i.e. the rate at which an incorrect base is incorporated into the daughter strand) is greater than 10⁻⁵, more preferably, greater than 10⁻⁴, even more preferably, greater than 10⁻³, and most preferably, greater than 10⁻². DNA polymerases having such high mutation rates are particularly advantageous for use in random mutagenesis experiments.

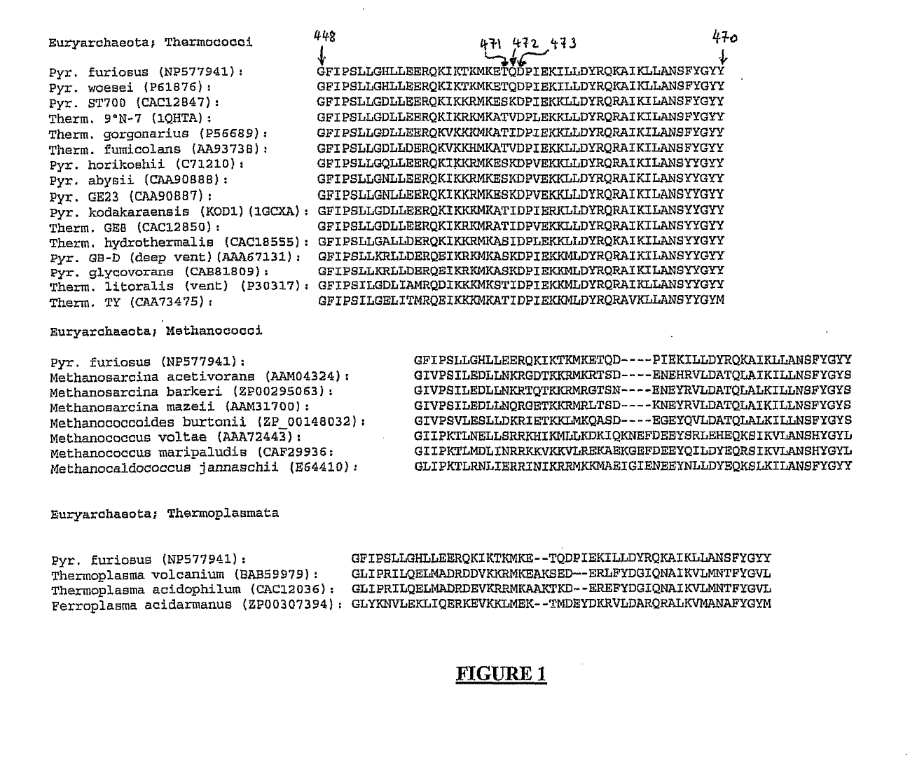

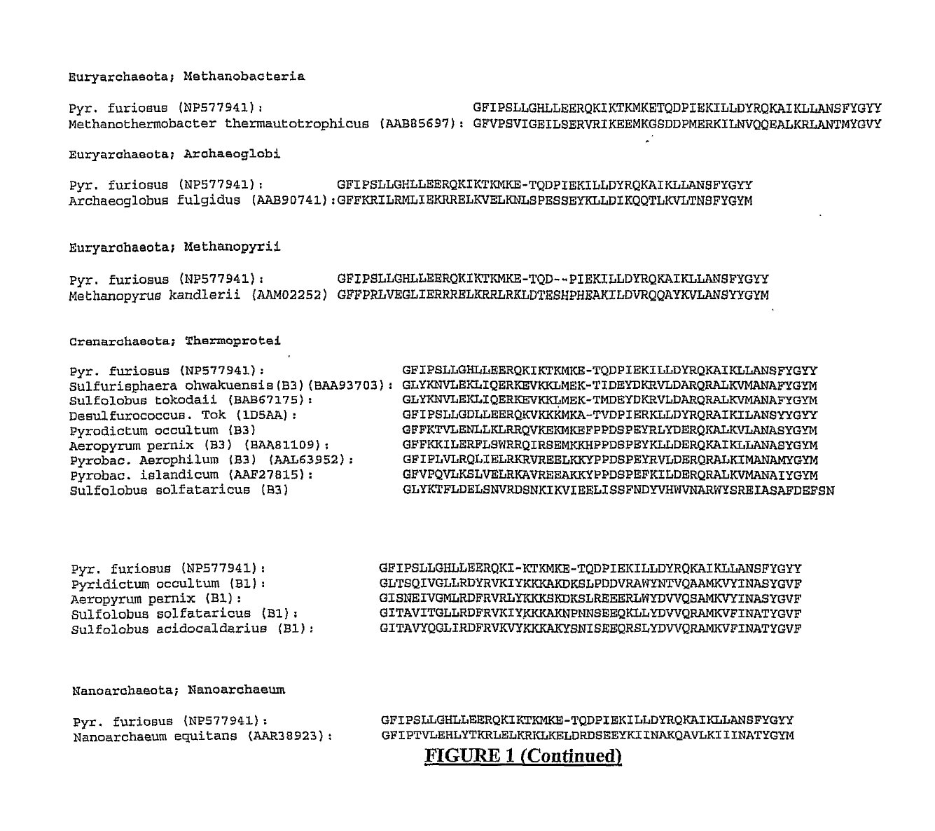

The variant archaeal DNA polymerase according to the first aspect may be a modification of an archaeal family DNA polymerase. It is preferred that the variant may be derived from any one of the archaeal family DNA polymerases illustrated in FIG. 1. However, it will be appreciated that the variant could also be derived from any other archaeal family DNA polymerase. It is especially preferred that the variant is derived from an archaeal family B DNA polymerase.

It is preferred that the variant polymerase is derived from a family independently selected from a group consisting of: Euryarchaeota; Crenarchaeota; and Nanoarchaeota.

It is especially preferred that the variant polymerase is derived from the Euryarchaeota family.

It is especially preferred that the variant polymerase is derived from a genus independently selected from a group consisting of: Thermococci; Methanococci; Thermoplasmata; Methanobacteria; Archaeoglobi; Methanopyrii; Thermoprotei; and Nanoarchaeum.

It is especially preferred that the variant is derived from the Thermococci genus.

It is preferred that the variant polymerase is derived from a species independently selected from a group consisting of: Pyrococcus furiosus (NP577941); Pyrococcus woesei (P61876); Pyrococcus ST700 (CAC12847); Thermococcus 9° N-7 (1QHTA); Thermococcus gorgoizarius (P56689); Thermococcus fumicolans (AA93738); Pyrococcus horikoshii (C71210); Pyrococcus abysii (CAA90888); Pyrococcus GE23 (CAA90887); Pyrococcus kodakaraensis (KOD1)(1GCXA); Thermococcus GE8 (CAC12850); Thermococcus hydrothermalis (CAC18555); Pyrococcus GB-D (deep vent)(AAA67131); Pyrococcus glycovorans (CAB81809); Thermococcus litoralis (vent) (P30317); and Thermococcus TY (CAA73475).

Preferably, the variant DNA polymerase is derived from Pyrococcus furiosus (Pfu-Pol).

The amino acid sequence of wild type Pfu-Pol used by the inventors comprises the following amino acid sequence:—

(SEQ ID NO.1)

MILDVDYITEEGKPVIRLFKKENGKFKIEHDRTFRPYIYALLRDDSKIEE
VKKITGERHGKIVRIVDVEKVEKKFLGKPITVWKLYLEHPQDVPTIREKV
REHPAVVDIFEYDIPFAKRYLIDKGLIPMEGEEELKILAFDIETLYHEGE
EFGKGPIIMISYADENEAKVITWKNIDLPYVEVVSSEREMIKRFLRIIRE
KDPDIIVTYNGDSFDFPYLAKRAEKLGIKLTIGRDGSEPKMQRIGDMTAV
EVKGRIHFDLYHVITRTINLPTYTLEAVYEAIFGKPKEKVYADEIAKAWE
SGENLERVAKYSMEDAKATYELGKEFLPMEIQLSRLVGQPLWDVSRSSTG
NLVEWFLLRKAYERNEVAPNKPSEEEYQRRLRESYTGGFVKEPEKGLWEN
IVYLDFRALYPSIIITHNVSPDTLNLEGCKNYDIAPQVGHKFCKDIPGFI
PSLLGHLLEERQKIKTKMKETQDPIEKILLDYRQKAIKLLANSFYGYYGY
AKARWYCKECAESVTAWGRKYIELVWKELEEKFGFKVLYIDTDGLYATIP
GGESEEIKKKALEFVKYINSKLPGLLELEYEGFYKRGFFVTKKRYAVIDE
EGKVITRGLEIVRRDWSEIAKETQARVLETILKHGDVEEAVRIVKEVIQK
LANYEIPPEKLAIYEQITRPLHEYKAIGPHVAVAKKLAAKGVKIKPGMVI
GYIVLRGDGPISNRAILAEEYDPKKHKYDAEYYIENQVLPAVLRILEGFG
YRKEDLRYQKTRQVGLTSWLNIKKS

Hence, preferably the variant DNA polymerase is derived from Pfu-Pol wild-type sequence. Preferred mutants according to the first aspect of the invention are variant forms of the wild-type sequence of Pfu-Pol (SEQ ID NO. 1).

Preferably, the variant polymerase according to the invention comprises at least one mutation in amino acid residues forming a loop (or hinge region) between two alpha helices, known as N alpha helix and 0 alpha helix of Pyrococcus furiosus DNA polymerase. Preferably, the loop in Pfu-Pol consists of three amino acid residues 471, 472 and 473. The loop in Pfu-Pol comprises TQD (underlined in SEQ ID NO. 1), where T is amino acid number 471, Q is amino acid number 472, and D is amino acid number 473.

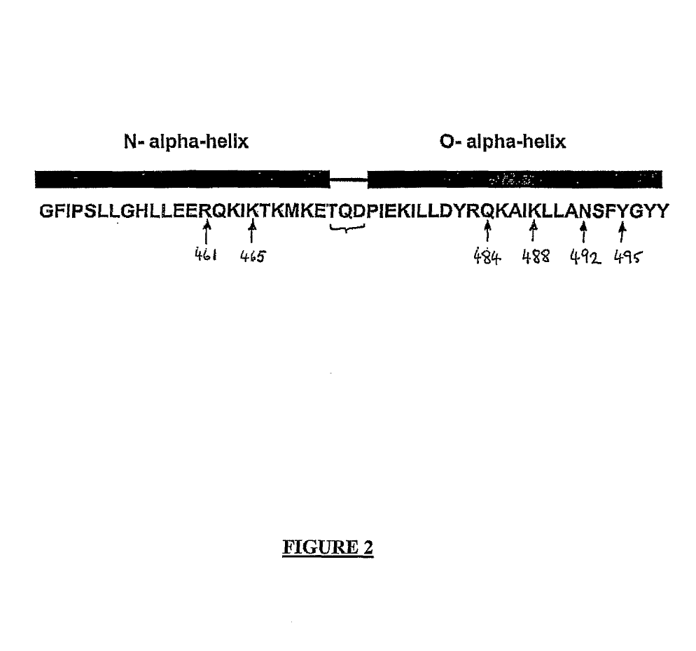

Hence, preferably, variants according to the invention comprise at least one modification in amino acids equivalent to 471, 472, and/or 473 of Pyrococcus furiosus DNA polymerase. It is preferred that the amino acids that form the two alpha helices that flank this loop comprise amino acids 448-470 (known as the N alpha-helix), and 474-498 (known as the 0 alpha-helix), and are illustrated in FIG. 2.

It will be appreciated that where amino acid residue numbers are provided in this specification, these refer to the equivalent position of residues when the sequence of the variant DNA polymerase according to the invention, is aligned with the enzyme from Pyrococcus furiosus, as is illustrated in FIG. 1. For example, the DNA polymerase from Methanococcus voltae (AAA72443), has four ‘additional’ amino acid residues (KNEF) immediately after the three amino acids (KIQ) forming the loop section between the N alpha-helix and the 0 alpha-helix. These three residues are not present in Pfu-Pol. Accordingly, the first residue of M. voltae DNA polymerase after these additional four residues, i.e. D, which does align with the sequence of Pfu-Pol, is numbered residue 474. The same applies to amino acid residues upstream and downstream of the loop region (417, 472, and 473).

The inventors were surprised that mutations in the loop (residues TQD) had a dramatic effect on the fidelity of the variant polymerase. Upon further investigation, they established that mutations in amino acid residues 471, 472 and 473 were particularly effective for mis-incorporation of bases in the replicated strand.

Hence, it is preferred that embodiments of the variant DNA polymerase may be formed by modification of amino acid residue 471 and/or 472 and/or 473 of the wild-type sequence of Pfu-Pol and such equivalent residues in other archaeal polymerases as illustrated in FIG. 1 (i.e. the amino acid residue equivalent to 471, 472 or 473 when aligned with Pfu-Pol). The modification may comprise a deletion or an insertion. However, preferably, the modification comprises a substitution.

The inventors have established that a Q472H modification of Pfu-Pol results in a high-fidelity DNA polymerase, and is therefore very useful for DNA sequencing, but they believe that it would not be useful in random mutagenesis protocols. While the inventors do not wish to be bound by any hypothesis, they believe it may be because histidine is a large amino acid. Accordingly, Q472H is therefore precluded from the first aspect of the invention. Hence, the variant DNA polymerase according to the first aspect is other than Q472H of Pyrococcus furiosus.

Accordingly, it is preferred that the modification at amino residue 471, 472 or 473 comprises a substitution with a small or constraining amino acid residue. Examples of suitable amino acids include glycine, alanine, proline, or serine.

Accordingly, it is preferred that an embodiment of the variant DNA polymerase comprises a modification of amino acid residue 471. The 471 modification may be a glycine or alanine substitution. Preferably, the modification is T471G or T471A.

It is especially preferred that an embodiment of the variant DNA polymerase comprises a modification of amino acid residue 472. Preferably, the modification at residue 472 comprises substitution with a small or constraining amino acid residue. The 472 modification may be a glycine, alanine or proline substitution. Preferably, the modification is Q472G, Q472A, or Q472P.

In addition, it is especially preferred that an embodiment of the variant DNA polymerase comprises a modification of amino acid residue 473. The 473 modification may be a glycine or alanine substitution. Preferably, the modification is D473G or D473A.

It is preferred that the variant DNA polymerase comprises a further modification to remove the 3′-5′ proof-reading exonuclease activity thereof. This would enable the proliferation of any mutations in the daughter DNA strands produced by replication. It is preferred that the further modification comprises a modified residue 215. Preferably, the 215 modification is an alanine substitution, and preferably a D215A modification, i.e. the Aspartic acid residue at position 215 of the wild-type sequence is modified to an Alanine residue.

It will be appreciated that the wild-type amino acid residue at, or equivalent to, position 471, 472, 473 and 215 in the variant polymerase according to the invention, may not be the same as the equivalent wild-type residue 471, 472, 473 and 215 in Pyrococcus furiosus. Accordingly, the skilled technician will appreciate that following an amino acid sequence alignment, the wild type amino acid residue (whatever that may be) in the variant polymerase equivalent to 471, 472, 473 and 215 in Pyrococcus furiosus, is modified. For example, referring to FIG. 1, for Pyrococcus woesei (P61876), the wild type residue 471 is S (serine). Hence, the variant polymerase for P. woesei may be S471G or S471A.

Preferred embodiments of the variant DNA polymerase in accordance with the invention comprise T471G & D215A; T471A & D215A; Q472G & D215A; Q472A & D215A; Q472P & D215A; D473G & D215A; or D473A & D215A modifications. Hence, the variant polymerase may comprise a double mutant.

It will be appreciated that the variant polymerase may comprise a mutant having a combination of modifications, for example, a 471 modification and/or a 472 modification and/or a 473 modification and/or a 215 modification. Examples of such mutants include Q472G & D473G & D215A; Q472A & D473A & D215A; Q472P & D473A & D215A, T471G & D215A & Q472G; T471A & D215A & D473A & Q472G, but it will be appreciated that other combinations of the modifications disclosed herein are contemplated.

The various variant polymerases were purified and their activity was compared with the wild type enzyme, which included only the modification to residue 215 (D215A), which acted as a control. However, the inventors were surprised to find that the variants according to the invention, having either a mutated residue 471 or 472 or 473, did incorporate an incorrect base during replication, suggesting much less fidelity than the wild-type enzyme.

The inventors noticed that mis-incorporation was most pronounced with the D473G modification, implying that this was the least accurate enzyme. Hence, it is especially preferred that the variant comprises a Glycine substitution at residue 473, preferably, a D473G modification. It is preferred that the variant further comprises an Alanine substitution at residue 215, preferably, a D215A modification.

The present invention therefore uses archaeal DNA polymerase from Pyrococcus furiosus (Pfu-Pol), which is an enzyme of higher thermostability and robustness compared to Taq-Pol. No changes to reaction conditions used for normal PCR are required given the rise to high yields of product. Higher levels of mutation are seen, and the distribution of mutations shows less bias than as compared to methods using Taq-Pol.

Hence, preferred Pfu-Pol variants in accordance with the invention comprise the following amino acid sequences:—

(a) Pfu-Pol Q472G

(SEQ ID NO.2)

MILDVDYITEEGKPVIRLFKKENGKFKIEHDRTFRPYIYALLRDDSKIEE
VKKITGERHGKIVRIVDVEKVEKKFLGKPITVWKLYLEHPQDVPTIREKV
REHPAVVDIFEYDIPFAKRYLIDKGLIPMEGEEELKILAFDIETLYHEGE
EFGKGPIIMISYADENEAKVITWKNIDLPYVEVVSSEREMIKRFLRIIRE
KDPDIIVTYNGDSFDFPYLAKRAEKLGIKLTIGRDGSEPKMQRIGDMTAV
EVKGRIHFDLYHVITRTINLPTYTLEAVYEAIFGKPKEKVYADEIAKAWE
SGENLERVAKYSMEDAKATYELGKEFLPMEIQLSRLVGQPLWDVSRSSTG
NLVEWFLLRKAYERNEVAPNKPSEEEYQRRLRESYTGGFVKEPEKGLWEN
IVYLDFRALYPSIIITHNVSPDTLNLEGCKNYDIAPQVGHKFCKDIPGFI
PSLLGHLLEERQKIKTKMKETGDPIEKILLDYRQKAIKLLANSFYGYYGY
AKARWYCKECAESVTAWGRKYIELVWKELEEKFGFKVLYIDTDGLYATIP
GGESEEIKKKALEFVKYINSKLPGLLELEYEGFYKRGFFVTKKRYAVIDE
EGKVITRGLEIVRRDWSEIAKETQARVLETILKHGDVEEAVRIVKEVIQK
LANYEIPPEKLAIYEQITRPLHEYKAIGPHVAVAKKLAAKGVKIKPGMVI
GYIVLRGDGPISNRAILAEEYDPKKHKYDAEYYIENQVLPAVLRILEGFG
YRKEDLRYQKTRQVGLTSWLNIKKS
(b) Pfu-Pol Q472A

(SEQ ID NO.3)

MILDVDYITEEGKPVIRLFKKENGKFKIEHDRTFRPYIYALLRDDSKIEE
VKKITGERHGKIVRIVDVEKVEKKFLGKPITVWKLYLEHPQDVPTIREKV
REHPAVVDIFEYDIPFAKRYLIDKGLIPMEGEEELKILAFDIETLYHEGE
EFGKGPIIMISYADENEAKVITWKNIDLPYVEVVSSEREMIKRFLRIIRE
KDPDIIVTYNGDSFDFPYLAKRAEKLGIKLTIGRDGSEPKMQRIGDMTAV
EVKGRIHFDLYHVITRTINLPTYTLEAVYEAIFGKPKEKVYADEIAKAWE
SGENLERVAKYSMEDAKATYELGKEFLPMEIQLSRLVGQPLWDVSRSSTG
NLVEWFLLRKAYERNEVAPNKPSEEEYQRRLRESYTGGFVKEPEKGLWEN
IVYLDFRALYPSIIITHNVSPDTLNLEGCKNYDIAPQVGHKFCKDIPGFI
PSLLGHLLEERQKIKTKMKETADPIEKILLDYRQKAIKLLANSFYGYYGY
AKARWYCKECAESVTAWGRKYIELVWKELEEKFGFKVLYIDTDGLYATIP
GGESEEIKKKALEFVKYINSKLPGLLELEYEGFYKRGFFVTKKRYAVIDE
EGKVITRGLEIVRRDWSEIAKETQARVLETILKHGDVEEAVRIVKEVIQK
LANYEIPPEKLAIYEQITRPLHEYKAIGPHVAVAKKLAAKGVKIKPGMVI
GYIVLRGDGPISNRAILAEEYDPKKHKYDAEYYIENQVLPAVLRILEGFG
YRKEDLRYQKTRQVGLTSWLNIKKS
(c) Pfu-Pol Q472P

(SEQ ID NO.4)

MILDVDYITEEGKPVIRLFKKENGKFKIEHDRTFRPYIYALLRDDSKIEE
VKKITGERHGKIVRIVDVEKVEKKFLGKPITVWKLYLEHPQDVPTIREKV
REHPAVVDIFEYDIPFAKRYLIDKGLIPMEGEEELKILAFDIETLYHEGE
EFGKGPIIMISYADENEAKVITWKNIDLPYVEVVSSEREMIKRFLRIIRE
KDPDIIVTYNGDSFDFPYLAKRAEKLGIKLTIGRDGSEPKMQRIGDMTAV
EVKGRIHFDLYHVITRTINLPTYTLEAVYEAIFGKPKEKVYADEIAKAWE
SGENLERVAKYSMEDAKATYELGKEFLPMEIQLSRLVGQPLWDVSRSSTG
NLVEWFLLRKAYERNEVAPNKPSEEEYQRRLRESYTGGFVKEPEKGLWEN
IVYLDFRALYPSIIITHNVSPDTLNLEGCKNYDIAPQVGHKFCKDIPGFI
PSLLGHLLEERQKIKTKMKETPDPIEKILLDYRQKAIKLLANSFYGYYGY
AKARWYCKECAESVTAWGRKYIELVWKELEEKFGFKVLYIDTDGLYATIP
GGESEEIKKKALEFVKYINSKLPGLLELEYEGFYKRGFFVTKKRYAVIDE
EGKVITRGLEIVRRDWSEIAKETQARVLETILKHGDVEEAVRIVKEVIQK
LANYEIPPEKLAIYEQITRPLHEYKAIGPHVAVAKKLAAKGVKIKPGMVI
GYIVLRGDGPISNRAILAEEYDPKKHKYDAEYYIENQVLPAVLRILEGFG
YRKEDLRYQKTRQVGLTSWLNIKKS
(d) Pfu-Pol D473A

(SEQ ID NO.5)

MILDVDYITEEGKPVIRLFKKENGKFKIEHDRTFRPYIYALLRDDSKIEE
VKKITGERHGKIVRIVDVEKVEKKFLGKPITVWKLYLEHPQDVPTIREKV
REHPAVVDIFEYDIPFAKRYLIDKGLIPMEGEEELKILAFDIETLYHEGE
EFGKGPIIMISYADENEAKVITWKNIDLPYVEVVSSEREMIKRFLRIIRE
KDPDIIVTYNGDSFDFPYLAKRAEKLGIKLTIGRDGSEPKMQRIGDMTAV
EVKGRIHFDLYHVITRTINLPTYTLEAVYEAIFGKPKEKVYADEIAKAWE
SGENLERVAKYSMEDAKATYELGKEFLPMEIQLSRLVGQPLWDVSRSSTG
NLVEWFLLRKAYERNEVAPNKPSEEEYQRRLRESYTGGFVKEPEKGLWEN
IVYLDFRALYPSIIITHNVSPDTLNLEGCKNYDIAPQVGHKFCKDIPGFI
PSLLGHLLEERQKIKTKMKETQAPIEKILLDYRQKAIKLLANSFYGYYGY
AKARWYCKECAESVTAWGRKYIELVWKELEEKFGFKVLYIDTDGLYATIP
GGESEEIKKKALEFVKYINSKLPGLLELEYEGFYKRGFFVTKKRYAVIDE
EGKVITRGLEIVRRDWSEIAKETQARVLETILKHGDVEEAVRIVKEVIQK
LANYEIPPEKLAIYEQITRPLHEYKAIGPHVAVAKKLAAKGVKIKPGMVI
GYIVLRGDGPISNRAILAEEYDPKKHKYDAEYYIENQVLPAVLRILEGFG
YRKEDLRYQKTRQVGLTSWLNIKKS
(e) Pfu-Pol D473G

(SEQ ID NO.6)

MILDVDYITEEGKPVIRLFKKENGKFKIEHDRTFRPYIYALLRDDSKIEE
VKKITGERHGKIVRIVDVEKVEKKFLGKPITVWKLYLEHPQDVPTIREKV
REHPAVVDIFEYDIPFAKRYLIDKGLIPMEGEEELKILAFDIETLYHEGE
EFGKGPIIMISYADENEAKVITWKNIDLPYVEVVSSEREMIKRFLRIIRE
KDPDIIVTYNGDSFDFPYLAKRAEKLGIKLTIGRDGSEPKMQRIGDMTAV
EVKGRIHFDLYHVITRTINLPTYTLEAVYEAIFGKPKEKVYADEIAKAWE
SGENLERVAKYSMEDAKATYELGKEFLPMEIQLSRLVGQPLWDVSRSSTG
NLVEWFLLRKAYERNEVAPNKPSEEEYQRRLRESYTGGFVKEPEKGLWEN
IVYLDFRALYPSIIITHNVSPDTLNLEGCKNYDIAPQVGHKFCKDIPGFI
PSLLGHLLEERQKIKTKMKETQGPIEKILLDYRQKAIKLLANSFYGYYGY
AKARWYCKECAESVTAWGRKYIELVWKELEEKFGFKVLYIDTDGLYATIP
GGESEEIKKKALEFVKYINSKLPGLLELEYEGFYKRGFFVTKKRYAVIDE
EGKVITRGLEIVRRDWSEIAKETQARVLETILKHGDVEEAVRIVKEVIQK
LANYEIPPEKLAIYEQITRPLHEYKAIGPHVAVAKKLAAKGVKIKPGMVI
GYIVLRGDGPISNRAILAEEYDPKKHKYDAEYYIENQVLPAVLRILEGFG
YRKEDLRYQKTRQVGLTSWLNIKKS
(1) Pfu-Pol T471G

(SEQ ID NO.7)

MILDVDYITEEGKPVIRLFKKENGKFKIEHDRTFRPYIYALLRDDSKIEE
VKKITGERHGKIVRIVDVEKVEKKFLGKPITVWKLYLEHPQDVPTIREKV
REHPAVVDIFEYDIPFAKRYLIDKGLIPMEGEEELKILAFDIETLYHEGE
EFGKGPIIMISYADENEAKVITWKNIDLPYVEVVSSEREMIKRFLRIIRE
KDPDIIVTYNGDSFDFPYLAKRAEKLGIKLTIGRDGSEPKMQRIGDMTAV
EVKGRIHFDLYHVITRTINLPTYTLEAVYEAIFGKPKEKVYADEIAKAWE
SGENLERVAKYSMEDAKATYELGKEFLPMEIQLSRLVGQPLWDVSRSSTG
NLVEWFLLRKAYERNEVAPNKPSEEEYQRRLRESYTGGFVKEPEKGLWEN
IVYLDFRALYPSIIITHNVSPDTLNLEGCKNYDIAPQVGHKFCKDIPGFI
PSLLGHLLEERQKIKTKMKEGQDPIEKILLDYRQKAIKLLANSFYGYYGY
AKARWYCKECAESVTAWGRKYIELVWKELEFKFGFKVLYIDTDGLYATIP
GGESEEIKKKALEFVKYINSKLPGLLELEYEGFYKRGFFVTKKRYAVIDE
EGKVITRGLEIVRRDWSEIAKETQARVLETILKHGDVEEAVRIVKEVIQK
LANYEIPPEKLAIYEQITRPLHEYKAIGPHVAVAKKLAAKGVKIKPGMVI
GYIVLRGDGPISNRAILAEEYDPKKHKYDAEYYIENQVLPAVLRILEGFG
YRKEDLRYQKTRQVGLTSWLNIKKS
(g) Pfu-Pol T471A

(SEQ ID NO.8)

MILDVDYITEEGKPVIRLFKKENGKFKIEHDRTFRPYIYALLRDDSKIEE
VKKITGERHGKIVRIVDVEKVEKKFLGKPITVWKLYLEHPQDVPTIREKV
REHPAVVDIFEYDIPFAKRYLIDKGLIPMEGEEELKILAFDIETLYHEGE
EFGKGPIIMISYADENEAKVITWKNIDLPYVEVVSSEREMIKRFLRIIRE
KDPDIIVTYNGDSFDFPYLAKRAEKLGIKLTIGRDGSEPKMQRIGDMTAV
EVKGRIHFDLYHVITRTINLPTYTLEAVYEAIFGKPKEKVYADEIAKAWE
SGENLERVAKYSMEDAKATYELGKEFLPMEIQLSRLVGQPLWDVSRSSTG
NLVEWFLLRKAYERNEVAPNKPSEEEYQRRLRESYTGGFVKEPEKGLWEN
IVYLDFRALYPSIIITHNVSPDTLNLEGCKNYDIAPQVGHKFCKDIPGFI
PSLLGHLLEERQKIKTKMKEAQDPIEKILLDYRQKAIKLLANSFYGYYGY
AKARWYCKECAESVTAWGRKYIELVWKELEEKFGFKVLYIDTDGLYATIP
GGESEEIKKKALEFVKYINSKLPGLLELEYEGFYKRGFFVTKKRYAVIDE
EGKVITRGLEIVRRDWSEIAKETQARVLETILKHGDVEEAVRIVKEVIQK
LANYEIPPEKLAIYEQITRPLHEYKAIGPHVAVAKKLAAKGVKIKPGMVI
GYIVLRGDGPISNRAILAEEYDPKKHKYDAEYYIENQVLPAVLRILEGFG
YRKEDLRYQKTRQVGLTSWLNIKKS

It will be appreciated that for each of the variant amino acid sequences provided above (SEQ ID NO. 2-8), residue 215 may be an A (Alanine) residue instead of a D (Aspartic acid) residue.

It will also be appreciated that equivalent amino acid residues (471, 472, 473, and 215) in any other archaeal DNA polymerases may be mutated, for example, in a DNA polymerase from a species independently selected from a group consisting of: Pyrococcus furiosus (NP577941); Pyrococcus woesei (P61876); Pyrococcus ST700 (CAC12847); Thermococcus 9° N-7 (1QHTA); Thermococcus gorgonarius (P56689); Thermococcus fumicolans (AA93738); Pyrococcus horikoshii (C71210); Pyrococcus abysii (CAA90888); Pyrococcus GE23 (CAA90887); Pyrococcus kodakaraensis (KOD1)(1GCXA); Thermococcus GE8 (CAC12850); Thermococcus hydrothermais (CAC18555); Pyrococcus GB-D (deep vent)(AAA67131); Pyrococcus glycovorans (CAB81809); Thermococcus litorals (vent) (P30317); and Thermococcus TY (CAA73475).

According to a second aspect there is provided a variant archaeal DNA polymerase comprising a modified amino acid sequence of a wild-type amino acid sequence, wherein the variant polymerase comprises a modification of amino acid residue 473.

The variant polymerase according to the second aspect may comprise an alanine substitution at residue 473, preferably, a D473A modification. It is especially preferred that the variant polymerase comprises a glycine substitution at residue 473, preferably, a D473G modification.

According to a third aspect there is provided a variant archaeal DNA polymerase comprising a modified amino acid sequence of a wild-type amino acid sequence, wherein the variant polymerase comprises a Q472G, Q472A, or Q472P modification.

According to a fourth aspect there is provided a variant archaeal DNA polymerase comprising a modified amino acid sequence of a wild-type amino acid sequence, wherein the variant polymerase comprises a modification of amino acid residue 471.

The variant polymerase according to the fourth aspect may comprise a glycine substitution at residue 471, preferably, a T471G modification. It is especially preferred that the variant polymerase comprises an alanine substitution at residue 471, preferably, a T471A modification.

It is preferred that the variant DNA polymerase according to either the second, third or fourth aspect comprises a further modification to remove the 3′-5′ proof-reading exonuclease activity thereof. Preferably, the variant archaeal DNA polymerase according to the second, third or fourth aspects comprises a modified residue 215, more preferably an alanine substitution at residue 215, for example, a D215A modification.

In a fifth aspect, the invention provides a nucleic acid encoding a variant DNA polymerase according to the first, second, third or fourth aspect of the invention or derivative or analogue thereof.

Preferred nucleic acids according to the fifth aspect of the invention may include:—

| (a) Pfu-Pol Q472G | |
|---|---|
| (SEQ ID NO.9) | |
| 1 | ATGATTTTAG ATGTGGATTA CATAACTGAA GAAGGAAAAC CTGTTATTAG GCTATTCAAA |
| 61 | AAAGAGAACG GAAAATTTAA GATAGAGCAT GATAGAACTT TTAGACCATA CATTTACGCT |
| 121 | CTTCTCAGGG ATGATTCAAA GATTGAAGAA GTTAAGAAAA TAACGGGGGA AAGGCATGGA |
| 181 | AAGATTGTGA GAATTGTTGA TGTAGAGAAG GTTGAGAAAA AGTTTCTCGG CAAGCCTATT |
| 241 | ACCGTGTGGA AACTTTATTT GGAACATCCC CAAGATGTTC CCACTATTAG AGAAAAAGTT |
| 301 | AGAGAACATC CAGCAGTTGT GGACATCTTC GAATACGATA TTCCATTTGC AAAGAGATAC |
| 361 | CTCATCGACA AAGGCCTAAT ACCAATGGAG GGGGAAGAAG AGCTAAAGAT TCTTGCCTTC |
| 421 | GATATAGAAA CCCTCTATCA CGAAGGAGAA GAGTTTGGAA AAGGCCCAAT TATAATGATT |
| 481 | AGTTATGCAG ATGAAAATGA AGCAAAGGTG ATTACTTGGA AAAACATAGA TCTTCCATAC |
| 541 | GTTGAGGTTG TATCAAGCGA GAGAGAGATG ATAAAGAGAT TTCTCAGGAT TATCAGGGAG |
| 601 | AAGGATCCTG ACATTATAGT TACTTATAAT GGAGACTCAT TCGACTTCCC ATATTTAGCG |
| 661 | AAAAGGGCAG AAAAACTTGG GATTAAATTA ACCATTGGAA GAGATGGAAG CGAGCCCAAG |
| 721 | ATGCAGAGAA TAGGCGATAT GACGGCTGTA GAAGTCAAGG GAAGAATACA TTTCGACTTG |
| 781 | TATCATGTAA TAACAAGGAC AATAAATCTC CCAACATACA CACTAGAGGC TGTATATGAA |
| 841 | GCAATTTTTG GAAAGCCAAA GGAGAAGGTA TACGCCGACG AGATAGCAAA AGCCTGGGAA |
| 901 | AGTGGAGAGA ACCTTGAGAG AGTTGCCAAA TACTCGATGG AAGATGCAAA GGCAACTTAT |
| 961 | GAACTCGGGA AAGAATTCCT TCCAATGGAA ATTCAGCTTT CAAGATTAGT TGGACAACCT |
| 1021 | TTATGGGATG TTTCAAGGTC AAGCACAGGG AACCTTGTAG AGTGGTTCTT ACTTAGGAAA |
| 1081 | GCCTACGAAA GAAACGAAGT AGCTCCAAAC AAGCCAAGTG AAGAGGAGTA TCAAAGAAGG |
| 1141 | CTCAGGGAGA GCTACACAGG TGGATTCGTT AAAGAGCCAG AAAAGGGGTT GTGGGAAAAC |
| 1201 | ATAGTATACC TAGATTTTAG AGCCCTATAT CCCTCGATTA TAATTACCCA CAATGTTTCT |
| 1261 | CCCGATACTC TAAATCTTGA GGGATGCAAG AACTATGATA TCGCTCCTCA AGTAGGCCAC |
| 1321 | AAGTTCTGCA AGGACATCCC TGGTTTTATA CCAAGTCTCT TGGGACATTT GTTAGAGGAA |
| 1381 | AGACAAAAGA TTAAGACAAA AATGAAGGAA ACTGGTGATC CTATAGAAAA AATACTCCTT |
| 1441 | GACTATAGAC AAAAAGCGAT AAAACTCTTA GCAAATTCTT TCTACGGATA TTATGGCTAT |
| 1501 | GCAAAAGCAA GATGGTACTG TAAGGAGTGT GCTGAGAGCG TTACTGCCTG GGGAAGAAAG |
| 1561 | TACATCGAGT TAGTATGGAA GGAGCTCGAA GAAAAGTTTG GATTTAAAGT CCTCTACATT |
| 1621 | GACACTGATG GTCTCTATGC AACTATCCCA GGAGGAGAAA GTGAGGAAAT AAAGAAAAAG |
| 1681 | GCTCTAGAAT TTGTAAAATA CATAAATTCA AAGCTCCCTG GACTGCTAGA GCTTGAATAT |
| 1741 | GAAGGGTTTT ATAAGAGGGG ATTCTTCGTT ACGAAGAAGA GGTATGCAGT AATAGATGAA |
| 1801 | GAAGGAAAAG TCATTACTCG TGGTTTAGAG ATAGTTAGGA GAGATTGGAG TGAAATTGCA |
| 1961 | AAAGAAACTC AAGCTAGAGT TTTGGAGACA ATACTAAAAC ACGGAGATGT TGAAGAAGCT |
| 2021 | GTGAGAATAG TAAAAGAAGT AATACAAAAG CTTGCCAATT ATGAAATTCC ACCAGAGAAG |
| 2081 | CTCGCAATAT ATGAGCAGAT AACAAGACCA TTACATGAGT ATAAGGCGAT AGGTCCTCAC |
| 2141 | GTAGCTGTTG CAAAGAAACT AGCTGCTAAA GGAGTTAAAA TAAAGCCAGG AATGGTAATT |
| 2201 | GGATACATAG TACTTAGAGG CGATGGTCCA ATTAGCAATA GGGCAATTCT AGCTGAGGAA |
| 2261 | TACGATCCCA AAAAGCACAA GTATGACGCA GAATATTACA TTGAGAACCA GGTTCTTCCA |
| 2321 | GCGGTACTTA GGATATTGGA GGGATTTGGA TACAGAAAGG AAGACCTCAG ATACCAAAAG |
| 2381 | ACAAGACAAG TCGGCCTAAC TTCCTGGCTT AACATTAAAA AATCCTAG |
| (b) Pfu-Pol Q472A | |
| (SEQ ID NO.10) | |
| 1 | ATGATTTTAG ATGTGGATTA CATAACTGAA GAAGGAAAAC CTGTTATTAG GCTATTCAAA |
| 61 | AAAGAGAACG GAAAATTTAA GATAGAGCAT GATAGAACTT TTAGACCATA CATTTACGCT |
| 121 | CTTCTCAGGG ATGATTCAAA GATTGAAGAA GTTAAGAAAA TAACGGGGGA AAGGCATGGA |
| 181 | AAGATTGTGA GAATTGTTGA TGTAGAGAAG GTTGAGAAAA AGTTTCTCGG CAAGCCTATT |
| 241 | ACCGTGTGGA AACTTTATTT GGAACATCCC CAAGATGTTC CCACTATTAG AGAAAAAGTT |
| 301 | AGAGAACATC CAGCAGTTGT GGACATCTTC GAATACGATA TTCCATTTGC AAAGAGATAC |
| 361 | CTCATCGACA AAGGCCTAAT ACCAATGGAG GGGGAAGAAG AGCTAAAGAT TCTTGCCTTC |
| 421 | GATATAGAAA CCCTCTATCA CGAAGGAGAA GAGTTTGGAA AAGGCCCAAT TATAATGATT |
| 481 | AGTTATGCAG ATGAAAATGA AGCAAAGGTG ATTACTTGGA AAAACATAGA TCTTCCATAC |
| 541 | GTTGAGGTTG TATCAAGCGA GAGAGAGATG ATAAAGAGAT TTCTCAGGAT TATCAGGGAG |
| 601 | AAGGATCCTG ACATTATAGT TACTTATAAT GGAGACTCAT TCGACTTCCC ATATTTAGCG |
| 661 | AAAAGGGCAG AAAAACTTGG GATTAAATTA ACCATTGGAA GAGATGGAAG CGAGCCCAAG |
| 721 | ATGCAGAGAA TAGGCGATAT GACGGCTGTA GAAGTCAAGG GAAGAATACA TTTCGACTTG |
| 781 | TATCATGTAA TAACAAGGAC AATAAATCTC CCAACATACA CACTAGAGGC TGTATATGAA |
| 841 | GCAATTTTTG GAAAGCCAAA GGAGAAGGTA TACGCCGACG AGATAGCAAA AGCCTGGGAA |
| 901 | AGTGGAGAGA ACCTTGAGAG AGTTGCCAAA TACTCGATGG AAGATGCAAA GGCAACTTAT |
| 961 | GAACTCGGGA AAGAATTCCT TCCAATGGAA ATTCAGCTTT CAAGATTAGT TGGACAACCT |
| 1021 | TTATGGGATG TTTCAAGGTC AAGCACAGGG AACCTTGTAG AGTGGTTCTT ACTTAGGAAA |
| 1081 | GCCTACGAAA GAAACGAAGT AGCTCCAAAC AAGCCAAGTG AAGAGGAGTA TCAAAGAAGG |
| 1141 | CTCAGGGAGA GCTACACAGG TGGATTCGTT AAAGAGCCAG AAAAGGGGTT GTGGGAAAAC |
| 1201 | ATAGTATACC TAGATTTTAG AGCCCTATAT CCCTCGATTA TAATTACCCA CAATGTTTCT |
| 1261 | CCCGATACTC TAAATCTTGA GGGATGCAAG AACTATGATA TCGCTCCTCA AGTAGGCCAC |
| 1321 | AAGTTCTGCA AGGACATCCC TGGTTTTATA CCAAGTCTCT TGGGACATTT GTTAGAGGAA |
| 1381 | AGACAAAAGA TTAAGACAAA AATGAAGGAA ACTGCTGATC CTATAGAAAA AATACTCCTT |
| 1441 | GACTATAGAC AAAAAGCGAT AAAACTCTTA GCAAATTCTT TCTACGGATA TTATGGCTAT |
| 1501 | GCAAAAGCAA GATGGTACTG TAAGGAGTGT GCTGAGAGCG TTACTGCCTG GGGAAGAAAG |
| 1561 | TACATCGAGT TAGTATGGAA GGAGCTCGAA GAAAAGTTTG GATTTAAAGT CCTCTACATT |
| 1621 | GACACTGATG GTCTCTATGC AACTATCCCA GGAGGAGAAA GTGAGGAAAT AAAGAAAAAG |
| 1681 | GCTCTAGAAT TTGTAAAATA CATAAATTCA AAGCTCCCTG GACTGCTAGA GCTTGAATAT |
| 1741 | GAAGGGTTTT ATAAGAGGGG ATTCTTCGTT ACGAAGAAGA GGTATGCAGT AATAGATGAA |
| 1801 | GAAGGAAAAG TCATTACTCG TGGTTTAGAG ATAGTTAGGA GAGATTGGAG TGAAATTGCA |
| 1961 | AAAGAAACTC AAGCTAGAGT TTTGGAGACA ATACTAAAAC ACGGAGATGT TGAAGAAGCT |
| 2021 | GTGAGAATAG TAAAAGAAGT AATACAAAAG CTTGCCAATT ATGAAATTCC ACCAGAGAAG |
| 2081 | CTCGCAATAT ATGAGCAGAT AACAAGACCA TTACATGAGT ATAAGGCGAT AGGTCCTCAC |
| 2141 | GTAGCTGTTG CAAAGAAACT AGCTGCTAAA GGAGTTAAAA TAAAGCCAGG AATGGTAATT |
| 2201 | GGATACATAG TACTTAGAGG CGATGGTCCA ATTAGCAATA GGGCAATTCT AGCTGAGGAA |
| 2261 | TACGATCCCA AAAAGCACAA GTATGACGCA GAATATTACA TTGAGAACCA GGTTCTTCCA |
| 2321 | GCGGTACTTA GGATATTGGA GGGATTTGGA TACAGAAAGG AAGACCTCAG ATACCAAAAG |
| 2381 | ACAAGACAAG TCGGCCTAAC TTCCTGGCTT AACATTAAAA AATCCTAG |
| (c) Pfu-Pol Q472P | |
| (SEQ ID NO.11) | |
| 1 | ATGATTTTAG ATGTGGATTA CATAACTGAA GAAGGAAAAC CTGTTATTAG GCTATTCAAA |
| 61 | AAAGAGAACG GAAAATTTAA GATAGAGCAT GATAGAACTT TTAGACCATA CATTTACGCT |
| 121 | CTTCTCAGGG ATGATTCAAA GATTGAAGAA GTTAAGAAAA TAACGGGGGA AAGGCATGGA |
| 181 | AAGATTGTGA GAATTGTTGA TGTAGAGAAG GTTGAGAAAA AGTTTCTCGG CAAGCCTATT |
| 241 | ACCGTGTGGA AACTTTATTT GGAACATCCC CAAGATGTTC CCACTATTAG AGAAAAAGTT |
| 301 | AGAGAACATC CAGCAGTTGT GGACATCTTC GAATACGATA TTCCATTTGC AAAGAGATAC |
| 361 | CTCATCGACA AAGGCCTAAT ACCAATGGAG GGGGAAGAAG AGCTAAAGAT TCTTGCCTTC |
| 421 | GATATAGAAA CCCTCTATCA CGAAGGAGAA GAGTTTGGAA AAGGCCCAAT TATAATGATT |
| 481 | AGTTATGCAG ATGAAAATGA AGCAAAGGTG ATTACTTGGA AAAACATAGA TCTTCCATAC |
| 541 | GTTGAGGTTG TATCAAGCGA GAGAGAGATG ATAAAGAGAT TTCTCAGGAT TATCAGGGAG |
| 601 | AAGGATCCTG ACATTATAGT TACTTATAAT GGAGACTCAT TCGACTTCCC ATATTTAGCG |
| 661 | AAAAGGGCAG AAAAACTTGG GATTAAATTA ACCATTGGAA GAGATGGAAG CGAGCCCAAG |
| 721 | ATGCAGAGAA TAGGCGATAT GACGGCTGTA GAAGTCAAGG GAAGAATACA TTTCGACTTG |
| 781 | TATCATGTAA TAACAAGGAC AATAAATCTC CCAACATACA CACTAGAGGC TGTATATGAA |
| 841 | GCAATTTTTG GAAAGCCAAA GGAGAAGGTA TACGCCGACG AGATAGCAAA AGCCTGGGAA |
| 901 | AGTGGAGAGA ACCTTGAGAG AGTTGCCAAA TACTCGATGG AAGATGCAAA GGCAACTTAT |
| 961 | GAACTCGGGA AAGAATTCCT TCCAATGGAA ATTCAGCTTT CAAGATTAGT TGGACAACCT |
| 1021 | TTATGGGATG TTTCAAGGTC AAGCACAGGG AACCTTGTAG AGTGGTTCTT ACTTAGGAAA |
| 1081 | GCCTACGAAA GAAACGAAGT AGCTCCAAAC AAGCCAAGTG AAGAGGAGTA TCAAAGAAGG |
| 1141 | CTCAGGGAGA GCTACACAGG TGGATTCGTT AAAGAGCCAG AAAAGGGGTT GTGGGAAAAC |
| 1201 | ATAGTATACC TAGATTTTAG AGCCCTATAT CCCTCGATTA TAATTACCCA CAATGTTTCT |
| 1261 | CCCGATACTC TAAATCTTGA GGGATGCAAG AACTATGATA TCGCTCCTCA AGTAGGCCAC |
| 1321 | AAGTTCTGCA AGGACATCCC TGGTTTTATA CCAAGTCTCT TGGGACATTT GTTAGAGGAA |
| 1381 | AGACAAAAGA TTAAGACAAA AATGAAGGAA ACTCCTGATC CTATAGAAAA AATACTCCTT |
| 1441 | GACTATAGAC AAAAAGCGAT AAAACTCTTA GCAAATTCTT TCTACGGATA TTATGGCTAT |
| 1501 | GCAAAAGCAA GATGGTACTG TAAGGAGTGT GCTGAGAGCG TTACTGCCTG GGGAAGAAAG |
| 1561 | TACATCGAGT TAGTATGGAA GGAGCTCGAA GAAAAGTTTG GATTTAAAGT CCTCTACATT |
| 1621 | GACACTGATG GTCTCTATGC AACTATCCCA GGAGGAGAAA GTGAGGAAAT AAAGAAAAAG |
| 1681 | GCTCTAGAAT TTGTAAAATA CATAAATTCA AAGCTCCCTG GACTGCTAGA GCTTGAATAT |
| 1741 | GAAGGGTTTT ATAAGAGGGG ATTCTTCGTT ACGAAGAAGA GGTATGCAGT AATAGATGCA |
| 1801 | GAAGGAAAAG TCATTACTCG TGGTTTAGAG ATAGTTAGGA GAGATTGGAG TGAAATTGCA |
| 1961 | AAAGAAACTC AAGCTAGAGT TTTGGAGACA ATACTAAAAC ACGGAGATGT TGAAGAAGCT |
| 2021 | GTGAGAATAG TAAAAGAAGT AATACAAAAG CTTGCCAATT ATGAAATTCC ACCAGAGAAG |
| 2081 | CTCGCAATAT ATGAGCAGAT AACAAGACCA TTACATGAGT ATAAGGCGAT AGGTCCTCAC |
| 2141 | GTAGCTGTTG CAAAGAAACT AGCTGCTAAA GGAGTTAAAA TAAAGCCAGG AATGGTAATT |
| 2201 | GGATACATAG TACTTAGAGG CGATGGTCCA ATTAGCAATA GGGCAATTCT AGCTGAGGAA |
| 2261 | TACGATCCCA AAAAGCACAA GTATGACGCA GAATATTACA TTGAGAACCA GGTTCTTCCA |
| 2321 | GCGGTACTTA GGATATTGGA GGGATTTGGA TACAGAAAGG AAGACCTCAG ATACCAAAAG |
| 2381 | ACAAGACAAG TCGGCCTAAC TTCCTGGCTT AACATTAAAA AATCCTAG |

(d) Pfu-Pol D473A
(SEQ ID NO.12)

| | |
|---|---|
| 1 | ATGATTTTAG ATGTGGATTA CATAACTGAA GAAGGAAAAC CTGTTATTAG GCTATTCAAA |
| 61 | AAAGAGAACG GAAAATTTAA GATAGAGCAT GATAGAACTT TTAGACCATA CATTTACGCT |
| 121 | CTTCTCAGGG ATGATTCAAA GATTGAAGAA GTTAAGAAAA TAACGGGGGA AAGGCATGGA |
| 181 | AAGATTGTGA GAATTGTTGA TGTAGAGAAG GTTGAGAAAA AGTTTCTCGG CAAGCCTATT |
| 241 | ACCGTGTGGA AACTTTATTT GGAACATCCC CAAGATGTTC CCACTATTAG AGAAAAAGTT |
| 301 | AGAGAACATC CAGCAGTTGT GGACATCTTC GAATACGATA TTCCATTTGC AAAGAGATAC |
| 361 | CTCATCGACA AAGGCCTAAT ACCAATGGAG GGGGAAGAAG AGCTAAAGAT TCTTGCCTTC |
| 421 | GATATAGAAA CCCTCTATCA CGAAGGAGAA GAGTTTGGAA AAGGCCCAAT TATAATGATT |
| 481 | AGTTATGCAG ATGAAAATGA AGCAAAGGTG ATTACTTGGA AAAACATAGA TCTTCCATAC |
| 541 | GTTGAGGTTG TATCAAGCGA GAGAGAGATG ATAAAGAGAT TTCTCAGGAT TATCAGGGAG |
| 601 | AAGGATCCTG ACATTATAGT TACTTATAAT GGAGACTCAT TCGACTTCCC ATATTTAGCG |
| 661 | AAAAGGGCAG AAAAACTTGG GATTAAATTA ACCATTGGAA GAGATGGAAG CGAGCCCAAG |
| 721 | ATGCAGAGAA TAGGCGATAT GACGGCTGTA GAAGTCAAGG GAAGAATACA TTTCGACTTG |
| 781 | TATCATGTAA TAACAAGGAC AATAAATCTC CCAACATACA CACTAGAGGC TGTATATGAA |
| 841 | GCAATTTTTG GAAAGCCAAA GGAGAAGGTA TACGCCGACG AGATAGCAAA AGCCTGGGAA |
| 901 | AGTGGAGAGA ACCTTGAGAG AGTTGCCAAA TACTCGATGG AAGATGCAAA GGCAACTTAT |
| 961 | GAACTCGGGA AAGAATTCCT TCCAATGGAA ATTCAGCTTT CAAGATTAGT TGGACAACCT |
| 1021 | TTATGGGATG TTTCAAGGTC AAGCACAGGG AACCTTGTAG AGTGGTTCTT ACTTAGGAAA |
| 1081 | GCCTACGAAA GAAACGAAGT AGCTCCAAAC AAGCCAAGTG AAGAGGAGTA TCAAAGAAGG |
| 1141 | CTCAGGGAGA GCTACACAGG TGGATTCGTT AAAGAGCCAG AAAAGGGGTT GTGGGAAAAC |
| 1201 | ATAGTATACC TAGATTTTAG AGCCCTATAT CCCTCGATTA TAATTACCCA CAATGTTTCT |
| 1261 | CCCGATACTC TAAATCTTGA GGGATGCAAG AACTATGATA TCGCTCCTCA AGTAGGCCAC |
| 1321 | AAGTTCTGCA AGGACATCCC TGGTTTTATA CCAAGTCTCT TGGGACATTT GTTAGAGGAA |
| 1381 | AGACAAAAGA TTAAGACAAA AATGAAGGAA ACTCAAGCTC CTATAGAAAA AATACTCCTT |
| 1441 | GACTATAGAC AAAAAGCGAT AAAACTCTTA GCAAATTCTT TCTACGGATA TTATGGCTAT |
| 1501 | GCAAAAGCAA GATGGTACTG TAAGGAGTGT GCTGAGAGCG TTACTGCCTG GGGAAGAAAG |
| 1561 | TACATCGAGT TAGTATGGAA GGAGCTCGAA GAAAAGTTTG GATTTAAAGT CCTCTACATT |
| 1621 | GACACTGATG GTCTCTATGC AACTATCCCA GGAGGAGAAA GTGAGGAAAT AAAGAAAAAG |
| 1681 | GCTCTAGAAT TTGTAAAATA CATAAATTCA AAGCTCCCTG GACTGCTAGA GCTTGAATAT |
| 1741 | GAAGGGTTTT ATAAGAGGGG ATTCTTCGTT ACGAAGAAGA GGTATGCAGT AATAGATGAA |
| 1801 | GAAGGAAAAG TCATTACTCG TGGTTTAGAG ATAGTTAGGA GAGATTGGAG TGAAATTGCA |
| 1961 | AAAGAAACTC AAGCTAGAGT TTTGGAGACA ATACTAAAAC ACGGAGATGT TGAAGAAGCT |
| 2021 | GTGAGAATAG TAAAAGAAGT AATACAAAAG CTTGCCAATT ATGAAATTCC ACCAGAGAAG |
| 2081 | CTCGCAATAT ATGAGCAGAT AACAAGACCA TTACATGAGT ATAAGGCGAT AGGTCCTCAC |
| 2141 | GTAGCTGTTG CAAAGAAACT AGCTGCTAAA GGAGTTAAAA TAAAGCCAGG AATGGTAATT |
| 2201 | GGATACATAG TACTTAGAGG CGATGGTCCA ATTAGCAATA GGGCAATTCT AGCTGAGGAA |
| 2261 | TACGATCCCA AAAAGCACAA GTATGACGCA GAATATTACA TTGAGAACCA GGTTCTTCCA |
| 2321 | GCGGTACTTA GGATATTGGA GGGATTTGGA TACAGAAAGG AAGACCTCAG ATACCAAAAG |
| 2381 | ACAAGACAAG TCGGCCTAAC TTCCTGGCTT AACATTAAAA AATCCTAG |

| (e) Pfu-Pol D473G | |
|---|---|
| (SEQ ID NO.13) | |
| 1 | ATGATTTTAG ATGTGGATTA CATAACTGAA GAAGGAAAAC CTGTTATTAG GCTATTCAAA |
| 61 | AAAGAGAACG GAAAATTTAA GATAGAGCAT GATAGAACTT TTAGACCATA CATTTACGCT |
| 121 | CTTCTCAGGG ATGATTCAAA GATTGAAGAA GTTAAGAAAA TAACGGGGGA AAGGCATGGA |
| 181 | AAGATTGTGA GAATTGTTGA TGTAGAGAAG GTTGAGAAAA AGTTTCTCGG CAAGCCTATT |
| 241 | ACCGTGTGGA AACTTTATTT GGAACATCCC CAAGATGTTC CCACTATTAG AGAAAAAGTT |
| 301 | AGAGAACATC CAGCAGTTGT GGACATCTTC GAATACGATA TTCCATTTGC AAAGAGATAC |
| 361 | CTCATCGACA AAGGCCTAAT ACCAATGGAG GGGGAAGAAG AGCTAAAGAT TCTTGCCTTC |
| 421 | GATATAGAAA CCCTCTATCA CGAAGGAGAA GAGTTTGGAA AAGGCCCAAT TATAATGATT |
| 481 | AGTTATGCAG ATGAAAATGA AGCAAAGGTG ATTACTTGGA AAAACATAGA TCTTCCATAC |
| 541 | GTTGAGGTTG TATCAAGCGA GAGAGAGATG ATAAAGAGAT TTCTCAGGAT TATCAGGGAG |
| 601 | AAGGATCCTG ACATTATAGT TACTTATAAT GGAGACTCAT TCGACTTCCC ATATTTAGCG |
| 661 | AAAAGGGCAG AAAAACTTGG GATTAAATTA ACCATTGGAA GAGATGGAAG CGAGCCCAAG |
| 721 | ATGCAGAGAA TAGGCGATAT GACGGCTGTA GAAGTCAAGG GAAGAATACA TTTCGACTTG |
| 781 | TATCATGTAA TAACAAGGAC AATAAATCTC CCAACATACA CACTAGAGGC TGTATATGAA |
| 841 | GCAATTTTTG GAAAGCCAAA GGAGAAGGTA TACGCCGACG AGATAGCAAA AGCCTGGGAA |
| 901 | AGTGGAGAGA ACCTTGAGAG AGTTGCCAAA TACTCGATGG AAGATGCAAA GGCAACTTAT |
| 961 | GAACTCGGGA AAGAATTCCT TCCAATGGAA ATTCAGCTTT CAAGATTAGT TGGACAACCT |
| 1021 | TTATGGGATG TTTCAAGGTC AAGCACAGGG AACCTTGTAG AGTGGTTCTT ACTTAGGAAA |
| 1081 | GCCTACGAAA GAAACGAAGT AGCTCCAAAC AAGCCAAGTG AAGAGGAGTA TCAAAGAAGG |
| 1141 | CTCAGGGAGA GCTACACAGG TGGATTCGTT AAAGAGCCAG AAAAGGGGTT GTGGGAAAAC |
| 1201 | ATAGTATACC TAGATTTTAG AGCCCTATAT CCCTCGATTA TAATTACCCA CAATGTTTCT |
| 1261 | CCCGATACTC TAAATCTTGA GGGATGCAAG AACTATGATA TCGCTCCTCA AGTAGGCCAC |
| 1321 | AAGTTCTGCA AGGACATCCC TGGTTTTATA CCAAGTCTCT TGGGACATTT GTTAGAGGAA |
| 1381 | AGACAAAAGA TTAAGACAAA AATGAAGGAA ACTCAAGGTC CTATAGAAAA AATACTCCTT |
| 1441 | GACTATAGAC AAAAAGCGAT AAAACTCTTA GCAAATTCTT TCTACGGATA TTATGGCTAT |
| 1501 | GCAAAAGCAA GATGGTACTG TAAGGAGTGT GCTGAGAGCG TTACTGCCTG GGGAAGAAAG |
| 1561 | TACATCGAGT TAGTATGGAA GGAGCTCGAA GAAAAGTTTG GATTTAAAGT CCTCTACATT |
| 1621 | GACACTGATG GTCTCTATGC AACTATCCCA GGAGGAGAAA GTGAGGAAAT AAAGAAAAAG |
| 1681 | GCTCTAGAAT TTGTAAAATA CATAAATTCA AAGCTCCCTG GACTGCTAGA GCTTGAATAT |
| 1741 | GAAGGGTTTT ATAAGAGGGG ATTCTTCGTT ACGAAGAAGA GGTATGCAGT AATAGATGAA |
| 1801 | GAAGGAAAAG TCATTACTCG TGGTTTAGAG ATAGTTAGGA GAGATTGGAG TGAAATTGCA |
| 1961 | AAAGAAACTC AAGCTAGAGT TTTGGAGACA ATACTAAAAC ACGGAGATGT TGAAGAAGCT |
| 2021 | GTGAGAATAG TAAAAGAAGT AATACAAAAG CTTGCCAATT ATGAAATTCC ACCAGAGAAG |
| 2081 | CTCGCAATAT ATGAGCAGAT AACAAGACCA TTACATGAGT ATAAGGCGAT AGGTCCTCAC |
| 2141 | GTAGCTGTTG CAAAGAAACT AGCTGCTAAA GGAGTTAAAA TAAAGCCAGG AATGGTAATT |
| 2201 | GGATACATAG TACTTAGAGG CGATGGTCCA ATTAGCAATA GGGCAATTCT AGCTGAGGAA |
| 2261 | TACGATCCCA AAAAGCACAA GTATGACGCA GAATATTACA TTGAGAACCA GGTTCTTCCA |
| 2321 | GCGGTACTTA GGATATTGGA GGGATTTGGA TACAGAAAGG AAGACCTCAG ATACCAAAAG |
| 2381 | ACAAGACAAG TCGGCCTAAC TTCCTGGCTT AACATTAAAA AATCCTAG |
| (f) Pfu-Pol T471G | |
| (SEQ ID NO.14) | |
| 1 | ATGATTTTAG ATGTGGATTA CATAACTGAA GAAGGAAAAC CTGTTATTAG GCTATTCAAA |
| 61 | AAAGAGAACG GAAAATTTAA GATAGAGCAT GATAGAACTT TTAGACCATA CATTTACGCT |
| 121 | CTTCTCAGGG ATGATTCAAA GATTGAAGAA GTTAAGAAAA TAACGGGGGA AAGGCATGGA |
| 181 | AAGATTGTGA GAATTGTTGA TGTAGAGAAA GTTGAGAAAA AGTTTCTCGG CAAGCCTATT |
| 241 | ACCGTGTGGA AACTTTATTT GGAACATCCC CAAGATGTTC CCACTATTAG AGAAAAAGTT |
| 301 | AGAGAACATC CAGCAGTTGT GGACATCTTC GAATACGATA TTCCATTTGC AAAGAGATAC |
| 361 | CTCATCGACA AAGGCCTAAT ACCAATGGAG GGGGAAGAAG AGCTAAAGAT TCTTGCCTTC |
| 421 | GATATAGAAA CCCTCTATCA CGAAGGAGAA GAGTTTGGAA AAGGCCCAAT TATAATGATT |
| 481 | AGTTATGCAG ATGAAAATGA AGCAAAGGTG ATTACTTGGA AAAACATAGA TCTTCCATAC |
| 541 | GTTGAGGTTG TATCAAGCGA GAGAGAGATG ATAAAGAGAT TTCTCAGGAT TATCAGGGAG |
| 601 | AAGGATCCTG ACATTATAGT TACTTATAAT GGAGACTCAT TCGACTTCCC ATATTTAGCG |
| 661 | AAAAGGGCAG AAAAACTTGG GATTAAATTA ACCATTGGAA GAGATGGAAG CGAGCCCAAG |
| 721 | ATGCAGAGAA TAGGCGATAT GACGGCTGTA GAAGTCAAGG GAAGAATACA TTTCGACTTG |
| 781 | TATCATGTAA TAACAAGGAC AATAAATCTC CCAACATACA CACTAGAGGC TGTATATGAA |
| 841 | GCAATTTTTG GAAAGCCAAA GGAGAAGGTA TACGCCGACG AGATAGCAAA AGCCTGGGAA |
| 901 | AGTGGAGAGA ACCTTGAGAG AGTTGCCAAA TACTCGATGG AAGATGCAAA GGCAACTTAT |
| 961 | GAACTCGGGA AAGAATTCCT TCCAATGGAA ATTCAGCTTT CAAGATTAGT TGGACAACCT |
| 1021 | TTATGGGATG TTTCAAGGTC AAGCACAGGG AACCTTGTAG AGTGGTTCTT ACTTAGGAAA |
| 1081 | GCCTACGAAA GAAACGAAGT AGCTCCAAAC AAGCCAAGTG AAGAGGAGTA TCAAAGAAGG |
| 1141 | CTCAGGGAGA GCTACACAGG TGGATTCGTT AAAGAGCCAG AAAAGGGGTT GTGGGAAAAC |
| 1201 | ATAGTATACC TAGATTTTAG AGCCCTATAT CCCTCGATTA TAATTACCCA CAATGTTTCT |
| 1261 | CCCGATACTC TAAATCTTGA GGGATGCAAG AACTATGATA TCGCTCCTCA AGTAGGCCAC |
| 1321 | AAGTTCTGCA AGGACATCCC TGGTTTTATA CCAAGTCTCT TGGGACATTT GTTAGAGGAA |
| 1381 | AGACAAAAGA TTAAGACAAA AATGAAGGAA GGTCAAGATC CTATAGAAAA AATACTCCTT |
| 1441 | GACTATAGAC AAAAAGCGAT AAAACTCTTA GCAAATTCTT TCTACGGATA TTATGGCTAT |
| 1501 | GCAAAAGCAA GATGGTACTG TAAGGAGTGT GCTGAGAGCG TTACTGCCTG GGGAAGAAAG |
| 1561 | TACATCGAGT TAGTATGGAA GGAGCTCGAA GAAAAGTTTG GATTTAAAGT CCTCTACATT |
| 1621 | GACACTGATG GTCTCTATGC AACTATCCCA GGAGGAGAAA GTGAGGAAAT AAAGAAAAAG |
| 1681 | GCTCTAGAAT TTGTAAAATA CATAAATTCA AAGCTCCCTG GACTGCTAGA GCTTGAATAT |
| 1741 | GAAGGGTTTT ATAAGAGGGG ATTCTTCGTT ACGAAGAAGA GGTATGCAGT AATAGATGAA |
| 1801 | GAAGGAAAAG TCATTACTCG TGGTTTAGAG ATAGTTAGGA GAGATTGGAG TGAAATTGCA |
| 1961 | AAAGAAACTC AAGCTAGAGT TTTGGAGACA ATACTAAAAC ACGGAGATGT TGAAGAAGCT |
| 2021 | GTGAGAATAG TAAAAGAAGT AATACAAAAG CTTGCCAATT ATGAAATTCC ACCAGAGAAG |
| 2081 | CTCGCAATAT ATGAGCAGAT AACAAGACCA TTACATGAGT ATAAGGCGAT AGGTCCTCAC |
| 2141 | GTAGCTGTTG CAAAGAAACT AGCTGCTAAA GGAGTTAAAA TAAAGCCAGG AATGGTAATT |
| 2201 | GGATACATAG TACTTAGAGG CGATGGTCCA ATTAGCAATA GGGCAATTCT AGCTGAGGAA |
| 2261 | TACGATCCCA AAAAGCACAA GTATGACGCA GAATATTACA TTGAGAACCA GGTTCTTCCA |
| 2321 | GCGGTACTTA GGATATTGGA GGGATTTGGA TACAGAAAGG AAGACCTCAG ATACCAAAAG |
| 2381 | ACAAGACAAG TCGGCCTAAC TTCCTGGCTT AACATTAAAA AATCCTAG |
| (g) Pfu-Pol T471A | |
| (SEQ ID NO.15) | |
| 1 | ATGATTTTAG ATGTGGATTA CATAACTGAA GAAGGAAAAC CTGTTATTAG GCTATTCAAA |
| 61 | AAAGAGAACG GAAAATTTAA GATAGAGCAT GATAGAACTT TTAGACCATA CATTTACGCT |
| 121 | CTTCTCAGGG ATGATTCAAA GATTGAAGAA GTTAAGAAAA TAACGGGGGA AAGGCATGGA |
| 181 | AAGATTGTGA GAATTGTTGA TGTAGAGAAG GTTGAGAAAA AGTTTCTCGG CAAGCCTATT |
| 241 | ACCGTGTGGA AACTTTATTT GGAACATCCC CAAGATGTTC CCACTATTAG AGAAAAAGTT |
| 301 | AGAGAACATC CAGCAGTTGT GGACATCTTC GAATACGATA TTCCATTTGC AAAGAGATAC |
| 361 | CTCATCGACA AAGGCCTAAT ACCAATGGAG GGGGAAGAAG AGCTAAAGAT TCTTGCCTTC |
| 421 | GATATAGAAA CCCTCTATCA CGAAGGAGAA GAGTTTGGAA AAGGCCCAAT TATAATGATT |
| 481 | AGTTATGCAG ATGAAAATGA AGCAAAGGTG ATTACTTGGA AAAACATAGA TCTTCCATAC |
| 541 | GTTGAGGTTG TATCAAGCGA GAGAGAGATG ATAAAGAGAT TTCTCAGGAT TATCAGGGAG |
| 601 | AAGGATCCTG ACATTATAGT TACTTATAAT GGAGACTCAT TCGACTTCCC ATATTTAGCG |
| 661 | AAAAGGGCAG AAAAACTTGG GATTAAATTA ACCATTGGAA GAGATGGAAG CGAGCCCAAG |
| 721 | ATGCAGAGAA TAGGCGATAT GACGGCTGTA GAAGTCAAGG GAAGAATACA TTTCGACTTG |
| 781 | TATCATGTAA TAACAAGGAC AATAAATCTC CCAACATACA CACTAGAGGC TGTATATGAA |
| 841 | GCAATTTTTG GAAAGCCAAA GGAGAAGGTA TACGCCGACG AGATAGCAAA AGCCTGGGAA |
| 901 | AGTGGAGAGA ACCTTGAGAG AGTTGCCAAA TACTCGATGG AAGATGCAAA GGCAACTTAT |
| 961 | GAACTCGGGA AAGAATTCCT TCCAATGGAA ATTCAGCTTT CAAGATTAGT TGGACAACCT |
| 1021 | TTATGGGATG TTTCAAGGTC AAGCACAGGG AACCTTGTAG AGTGGTTCTT ACTTAGGAAA |
| 1081 | GCCTACGAAA GAAACGAAGT AGCTCCAAAC AAGCCAAGTG AAGAGGAGTA TCAAAGAAGG |
| 1141 | CTCAGGGAGA GCTACACAGG TGGATTCGTT AAAGAGCCAG AAAAGGGGTT GTGGGAAAAC |
| 1201 | ATAGTATACC TAGATTTTAG AGCCCTATAT CCCTCGATTA TAATTACCCA CAATGTTTCT |
| 1261 | CCCGATACTC TAAATCTTGA GGGATGCAAG AACTATGATA TCGCTCCTCA AGTAGGCCAC |
| 1321 | AAGTTCTGCA AGGACATCCC TGGTTTTATA CCAAGTCTCT TGGGACATTT GTTAGAGGAA |
| 1381 | AGACAAAAGA TTAAGACAAA AATGAAGGAA GCTCAAGATC CTATAGAAAA AATACTCCTT |
| 1441 | GACTATAGAC AAAAAGCGAT AAAACTCTTA GCAAATTCTT TCTACGGATA TTATGGCTAT |
| 1501 | GCAAAAGCAA GATGGTACTG TAAGGAGTGT GCTGAGAGCG TTACTGCCTG GGGAAGAAAG |
| 1561 | TACATCGAGT TAGTATGGAA GGAGCTCGAA GAAAAGTTTG GATTTAAAGT CCTCTACATT |
| 1621 | GACACTGATG GTCTCTATGC AACTATCCCA GGAGGAGAAA GTGAGGAAAT AAAGAAAAAG |
| 1681 | GCTCTAGAAT TTGTAAAATA CATAAATTCA AAGCTCCCTG GACTGCTAGA GCTTGAATAT |
| 1741 | GAAGGGTTTT ATAAGAGGGG ATTCTTCGTT ACGAAGAAGA GGTATGCAGT AATAGATGAA |
| 1801 | GAAGGAAAAG TCATTACTCG TGGTTTAGAG ATAGTTAGGA GAGATTGGAG TGAAATTGCA |
| 1961 | AAAGAAACTC AAGCTAGAGT TTTGGAGACA ATACTAAAAC ACGGAGATGT TGAAGAAGCT |
| 2021 | GTGAGAATAG TAAAAGAAGT AATACAAAAG CTTGCCAATT ATGAAATTCC ACCAGAGAAG |
| 2081 | CTCGCAATAT ATGAGCAGAT AACAAGACCA TTACATGAGT ATAAGGCGAT AGGTCCTCAC |
| 2141 | GTAGCTGTTG CAAAGAAACT AGCTGCTAAA GGAGTTAAAA TAAAGCCAGG AATGGTAATT |
| 2201 | GGATACATAG TACTTAGAGG CGATGGTCCA ATTAGCAATA GGGCAATTCT AGCTGAGGAA |
| 2261 | TACGATCCCA AAAAGCACAA GTATGACGCA GAATATTACA TTGAGAACCA GGTTCTTCCA |
| 2321 | GCGGTACTTA GGATATTGGA GGGATTTGGA TACAGAAAGG AAGACCTCAG ATACCAAAAG |
| 2381 | ACAAGACAAG TCGGCCTAAC TTCCTGGCTT AACATTAAAA AATCCTAG |

For the nucleic acid sequences given above (SEQ ID NO. 9 to SEQ ID NO:15), the three codons at position 643-646 (underlined and in bold) are GAC and encode the D215 modification. However, it will be appreciated that these codons may also be GCT or GCC or GCA or GCG, all of which code for A215 giving the D215A exonuclease variant. It will be realised that the codons at positions 471, 472 and 473 (underlined and in bold), which encode T471G, T471A, Q472G, Q472A, Q472P, D473A and D473G may, within the confines of the genetic code, be replaced with other codons specifying the same amino acid.

The nucleic acid may be an isolated or purified nucleic acid sequence. The nucleic acid sequence may be a DNA sequence. The nucleic acid sequence may further comprise elements capable of controlling and/or enhancing its expression. The nucleic acid molecule may be contained within a suitable vector to form a recombinant vector. The vector may for example be a plasmid, cosmid or phage.

Recombinant vectors may also include other functional elements. For instance, recombinant vectors can be designed such that the vector will autonomously replicate in the cell. In this case elements that induce nucleic acid replication may be required in the recombinant vector. Alternatively, the recombinant vector may be designed such that the vector and recombinant nucleic acid molecule integrates into the genome of a cell. In this case nucleic acid sequences, which favour targeted integration (e.g. by homologous recombination) are desirable. Recombinant vectors may also have DNA coding for genes that may be used as selectable markers in the cloning process. The recombinant vector may also further comprise a promoter or regulator to control expression of the gene as required.

The variant DNA polymerase in accordance with the first, second, third or fourth aspects are preferably adapted to mis-incorporate a greater number of incorrect bases than compared to a wild-type archaeal DNA polymerase, and therefore may be used in a random mutagenesis protocol.

Hence, according to a sixth aspect of the invention, there is provided use of a variant archaeal DNA polymerase according to the first, second, third or fourth aspects of the invention, in random mutagenesis protocols.

According to a seventh aspect of the present invention there is provided a method of causing random mutations in DNA comprising the steps of:—(i) denaturing a double strand of DNA by heating a solution containing the DNA, free oligonucleotides, primers and a variant archaeal DNA polymerase according to the first, second, third or fourth aspect of the invention; (ii) reducing the temperature of the solution to effect annealing of the primer and the DNA and (iii) allowing extension of the DNA strand by the variant polymerase.

The increased rate of mutation exhibited by variant polymerases used according to the sixth or seventh aspects of the invention means that they are surprisingly effective at introducing random mutations in to a DNA molecule being investigated, and hence protein it encodes. This radically increases the number of mutants produced, which enables an investigator to perform valuable structure/function experiments.

The variant polymerases according to the invention are particularly useful in random mutagenesis experiments because they are thermally stable, but have low fidelity, and have no 3′-5′ proof-reading ability. The DNA to be mutated may encode a protein being investigated, for example, an enzyme.

It will be appreciated that the random mutagenesis protocol may be any experimental procedure that involves at least one cycle of replication with the variant polymerase. It is preferred that the protocol involves multiple cycles of replication and that the protocol is based on the Polymerase Chain Reaction.

A preferred protocol for carrying out PCR utilising variant polymerases according to the present invention is as follows: PCR may carried out under the following conditions: 100 μl volume, 20 mM Tris-HCl pH 8.8, 10 mM KCl, 10 mM (NH₄)₂ SO₄, 2 mM MgSO₄, 0.1% Triton X100, 100 μg/ml BSA, 250 μM each of dATP, dGTP, dCTP and dTTP, 2.5 units DNA polymerase overlayed with 40 μl of mineral oil (1 unit of polymerase is defined as amount of enzyme that incorporates 10 mmol of dATP into acid-precipitable material using an activated calf-thymus DNA-based assay (4) in 30 min at 72° C.). 5 ng of template DNA to be amplified is used and the concentration of the forward and reverse primers (each 18 bases in length) is 0.3 μM. Each PCR consisted of 30 cycles of 1 min at 95° C., 2 min at 52° C. and 4.5 min at 72° C.

A skilled person will appreciate that a molecular biologist will be able to adapt standard PCR protocols for use with the variant polymerases. Individual protocols, and primers used therein, should also be adapted in the light of the template nucleic acid, which is to be mutated.

According to an eighth aspect of the present invention there is provided a kit useful for random mutagenesis protocols comprising a variant archaeal DNA polymerase according to the first, second, third or fourth aspects of the invention; and optionally buffers and free bases.

Kits designed for mutagenesis of specific proteins may also include DNA for mutation and primers for annealing thereto.

Based on their preliminary findings researching the use of mutated archaeal DNA polymerases, the inventors decided to investigate whether there were similar or even homologous genes encoding DNA polymerases or regions thereof, in any higher organisms, which could be mutated to produce an enzyme which is adapted to mis-incorporate a greater number of incorrect bases than compared to an equivalent wild-type DNA polymerase.

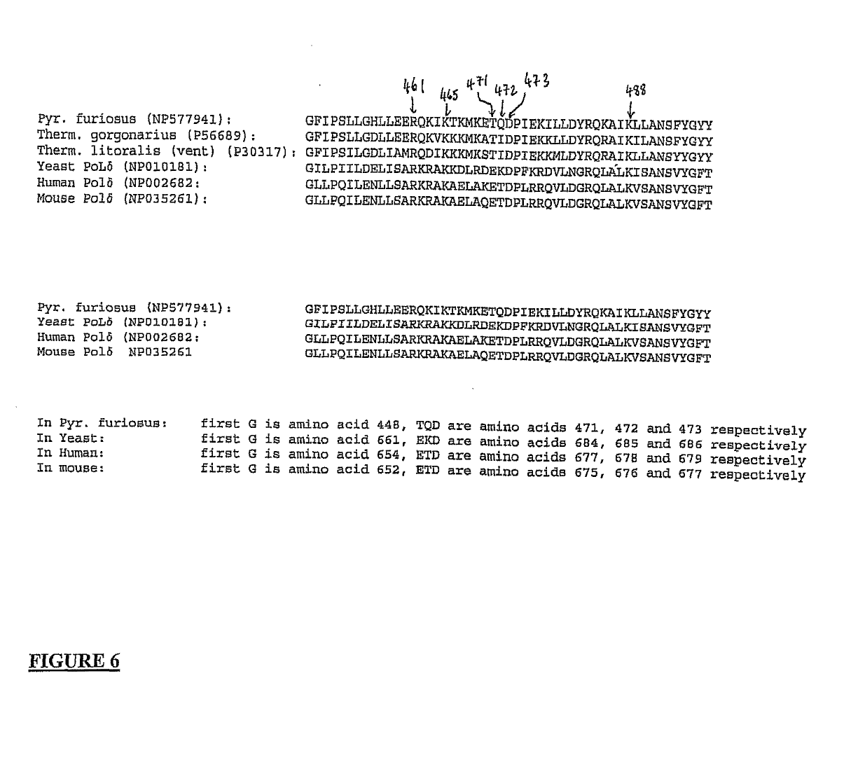

To their surprise, the inventors discovered a very good line up (based on amino acid sequences) between the helix-loop-helix region (i.e. equivalent to amino acid residues 248-298 of Pyrococcus furiosus DNA polymerase) of archaea, and eukaryotic DNA polymerase delta. This is illustrated in FIG. 6.

According to a ninth aspect of the invention, there is provided a variant DNA polymerase comprising a modified amino acid sequence of a wild-type amino acid sequence, the variant comprising at least one modification in amino acids equivalent to residues 471, 472, or 473 of Pyrococcus furiosus DNA polymerase, wherein during DNA replication, the variant polymerase is adapted to mis-incorporate a greater number of incorrect bases than compared to a wild-type DNA polymerase.

Preferably, a loop or hinge region of the variant DNA polymerase comprises the three amino acids 471, 472, and 473, when aligned with the sequence of the Pyrococcus furiosus DNA polymerase. It is preferred that the loop is between the two alpha-helices represented by equivalent to amino acids 448-470 and 474-498 in the Pyrococcus furiosus DNA polymerase. The amino acids that form the two alpha helices that flank this loop comprise amino acids 448-470 (known as the N alpha-helix in Pfu-pol), and 474-498 (known as the O alpha-helix in Pfu-pol), and are illustrated in FIG. 6.

By the term “equivalent to”, we mean those amino acid residues of the variant DNA polymerase according to the invention, which align with amino acid residues 471, 472, and 473 of Pyrococcus furiosus DNA polymerase.

The variant DNA polymerase may be a DNA polymerase delta (a). The variant DNA polymerase may be derived from a eukaryote or prokaryote.

It will be appreciated that eukaryotic DNA polymerases may be multi-subunit enzymes. Hence, the functional enzyme may consist of more than one subunit, each subunit being encoded by more than one gene. Accordingly, by DNA polymerase referred to in the ninth aspect, we are preferably referring to a catalytic subunit of the DNA polymerase enzyme. It will also be appreciated that the two helices in eukaryotic polymerases referred to in FIG. 6 are those two helices, which are equivalent to helices N and O in Pfu-pol.

For example, the variant DNA polymerase may be derived from a mammal such as a human, or a mouse, or a fungus, such as yeast.

The accession number for the wild-type S. cerevisiae DNA polymerase is NP010181. The accession number for the wild-type mouse DNA polymerase is NPO035261. The accession number for the wild-type human DNA polymerase is NP002682. The skilled technician would appreciate how to mutate the amino acids 471, 472 and/or 473 in these enzymes.

In particular, the inventors noted that the key D (aspartic acid) residue at position 473 of Pyrococcus furiosus is conserved. It is thought that Polymerase delta is the main DNA replicating enzyme in eukaryotes. Although these polymerases are unlikely to be useful for PCR-based methods, because these are non-thermostable, a low fidelity eukaryotic variant may have other uses e.g. in vivo or in vitro random mutagenesis.

Hence, it is preferred that embodiments of the variant DNA polymerase may be formed by modification of amino acid residue 471 and/or 472 and/or 473 equivalent to the wild-type sequence of Pfu-Pol and such equivalent residues in other polymerases, as illustrated in FIG. 6 (i.e. the amino acid residue equivalent to 471, 472 or 473 when aligned with Pfu-Pol). The modification may comprise a deletion or an insertion. However, preferably, the modification comprises a substitution.

It is preferred that the modification at amino residue 471, 472 or 473 comprises a substitution with a small or constraining amino acid residue. Examples of suitable amino acids include glycine, alanine, proline, or serine.

Accordingly, it is preferred that an embodiment of the variant DNA polymerase comprises a modification of amino acid residue 471. The 471 modification may be a glycine or alanine substitution. The modification may be T471G or T471A. It is especially preferred that an embodiment of the variant DNA polymerase comprises a modification of amino acid residue 472. The 472 modification may be a glycine, alanine or proline substitution. Preferably, the modification is Q472G, Q472A, or Q472P. It is especially preferred that an embodiment of the variant DNA polymerase comprises a modification of amino acid residue 473. The 473 modification may be a glycine or alanine substitution. Preferably, the modification is D473G or D473A.

It will be appreciated that where amino acid residue numbers are provided in this specification, these refer to the equivalent position of residues when the amino acid sequence of the variant DNA polymerase according to the ninth aspect of the invention, is aligned with the amino acid sequence of the enzyme from Pyrococcus furiosus, as is illustrated in FIG. 6. However, as illustrated in FIG. 6, the actual amino acid numbers for the variant DNA polymerase will differ due to differences in the sequence upstream of the hinge region. For example:—

In Pyrococcus furiosus: the first G is amino acid 448, TQD are amino acids 471, 472 and 473, respectively. In Yeast: the first G is amino acid 661, EKD are amino acids 684, 685 and 686 respectively. In Human: the first G is amino acid 654, ETD are amino acids 677, 678 and 679, respectively. In mouse: the first G is amino acid 652, ETD are amino acids 675, 676 and 677, respectively.

In Pyrococcus furiosus, the mutation D215A abolishes 3′ to 5′ proof reading exonuclease activity. Hence, the variant DNA polymerase may comprise a mutation at amino acid equivalent to residue 215 of Pyrococcus furiosus DNA polymerase. The variant DNA polymerase may comprise an alanine substitution, for example, a D215A modification. The equivalent mutations in yeast, human and mouse are D407A, D402A and D400A, respectively.

It will be appreciated that the wild-type amino acid residue at, or equivalent to, position 471, 472, 473 and 215 in the variant polymerase according to the invention, may not be the same as the equivalent wild-type residue 471, 472, 473 and 215 in Pyrococcus furiosus. Accordingly, the skilled technician will appreciate that following an amino acid sequence alignment, the wild type amino acid residue (whatever that may be) in the variant polymerase equivalent to 471, 472, 473 and 215 in Pyrococcus furiosus, is modified. For example, referring to FIG. 6, for mouse, the wild type residue 471 is E (glutamic acid). Hence, the variant polymerase for P. woesei may be E471G or E471A.

Accordingly, it may be possible to replace yeast polymerase delta with, for example, a D473 variant (a low fidelity variant), and use it to mutate genes and hence proteins in vivo. Alternatively, a eukaryotic variant DNA polymerase could be used in the preparation of yeast or mammalian cells having novel properties. The low-fidelity variant polymerase could be introduced into a eukaryotic cell, and subsequent daughter cells could be selected for properties such as thermostability, or increased production of useful metabolites etc. A human variant polymerase may be used for in vitro experiments to produce mutated daughter DNA strands, which may then be introduced into cells in vivo.

Hence, according to a tenth aspect of the invention, there is provided use of a variant DNA polymerase according to the ninth aspect of the invention, in random mutagenesis protocols.

According to an eleventh aspect there is provided a method of causing random mutations in DNA comprising the steps of:—(i) denaturing a double strand of DNA by heating a solution containing the DNA, free oligonucleotides, primers and a variant DNA polymerase according to the ninth aspect of the invention; (ii) reducing the temperature of the solution to effect annealing of the primer and the DNA and (iii) allowing extension of the DNA strand by the variant polymerase.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

FIG. 1 illustrates an amino acid sequence alignment of a number of archaeal DNA polymerases corresponding to residues 248-298 of Pyrococcus furiosus polymerase;

FIG. 2 illustrates the amino acid sequence of residues 248-298 of Pyrococcus furiosus DNA polymerase;

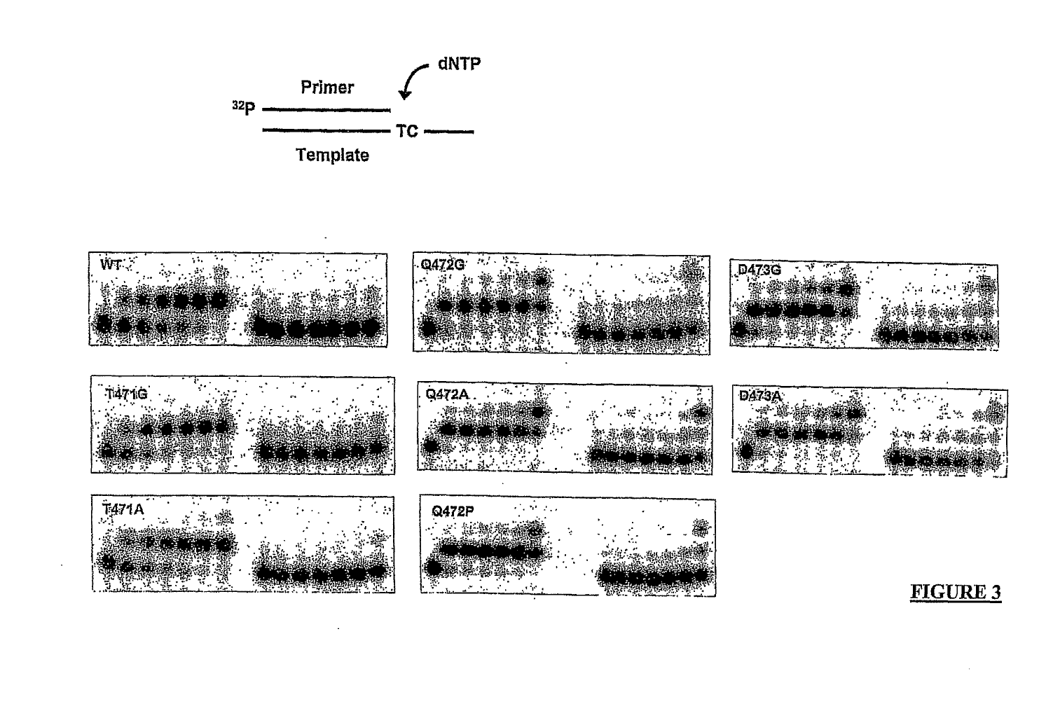

FIG. 3 shows the results of a single-base primer extension assay using embodiments of a variant DNA polymerase according to the invention;

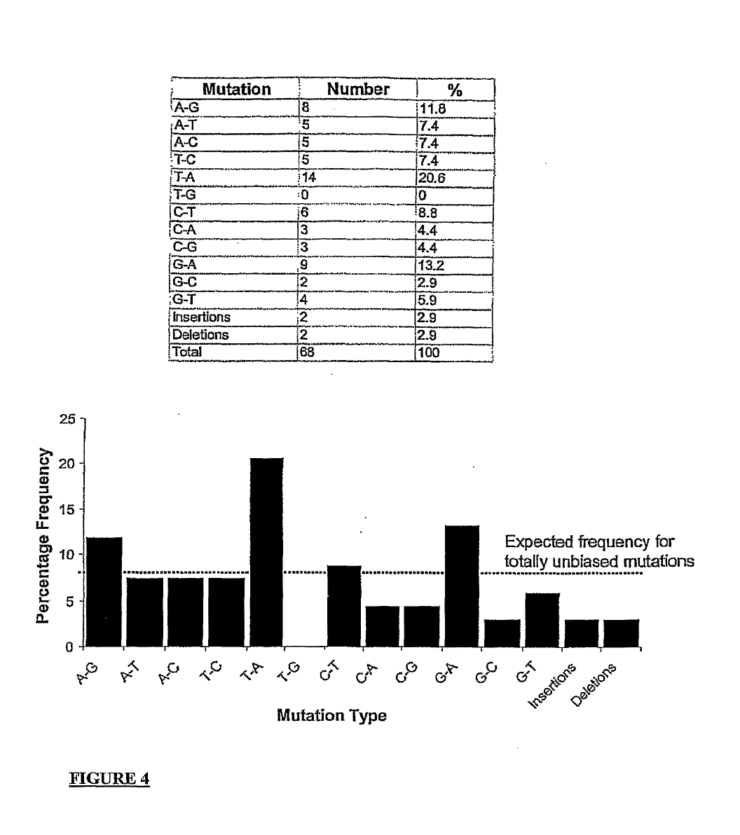

FIG. 4 shows a mutation spectrum produced by a variant polymerase Pfu-Pol(D215A, D473G) according to the invention;

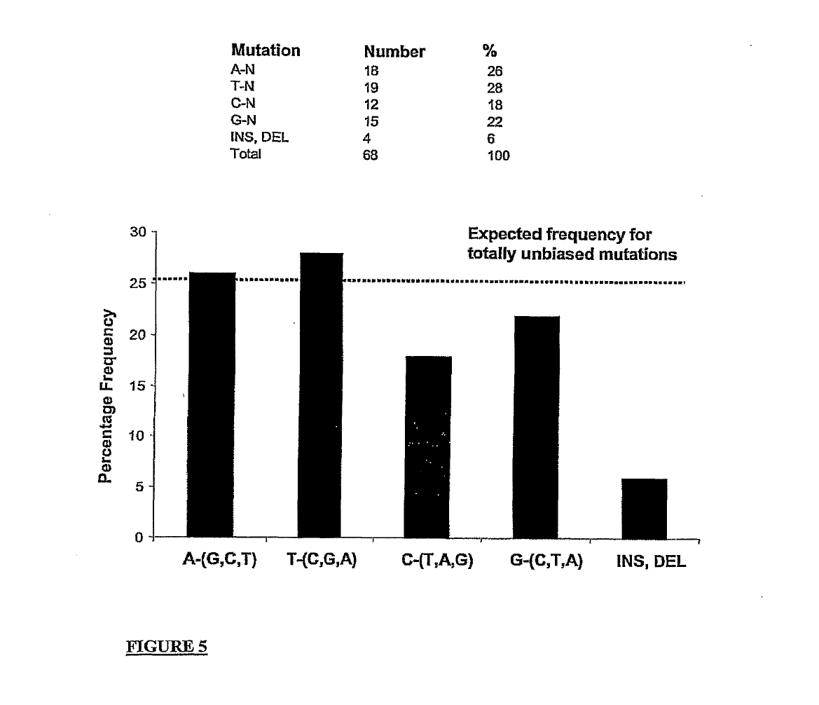

FIG. 5 represents Bias Indication for the variant polymerase; and

FIG. 6 illustrates an amino acid sequence alignment of three archaeal DNA polymerases, with homologous yeast, human and mouse enzymes.

### EXAMPLE

Production and Purification of DNA Polymerase Variants

Site directed mutagenesis, to produce the variant polymerises in accordance with the invention, was achieved using overlap extension PCR (Ho, S, N., Hunt, N. D., Horton, R. M., Pullen, J. K. and Pease, L. R. (1989) Gene 77, 51-59) using Pfu-Turbo (Stratagene) or using QuickChange® Site-Directed Mutagenesis Kit (Stratagene).

Mutagenesis was performed on pET17b (Pfu-Pol), a protein expression plasmid containing the gene coding for Pfu-Pol under the control of the bacteriophage T7 promoter. Full details of this plasmid have been described (Evans, S. J., Fogg, M. J., Mamone, A., Davis, M., Pearl, L. H. and Connolly, B. A. (2000) Nucleic Acids Research 5, 1059-1066). Following mutagenesis by overlap extension PCR the resulting plasmids were used to transform E. coli BL21 (pLysS). Cells, containing plasmids with a variant polymerase, were grown to OD₆₀₀ of 0.8-1, and protein expression induced by addition of IPTG (1 mM final) and cells incubated at 30° C. for a further 18 hours. Cells were pelleted by centrifugation (15 min, 4000×g), resuspended in 20 mM Tris-HCl (pH 8.0), 100 mM NaCl, 0.1 mM PMSF, 0.1 mM benzamidine and sonicated on ice. Cell debris was cleared by centrifugation (45 min, 30000×g), the lysate incubated with DNaseI (˜20 U, 37° C., 30 mins), and heated to 85° C. for 20 mins.

Denatured host proteins were removed by centrifugation (45 mins, 30000×g) and the supernatant was loaded onto a 20 ml DEAE-Sephacel (Amersham Biosciences) column, equilibrated to 20 mM Tris-HCl (pH 8.0), 100 mM NaCl. The flow through was applied directly to a 20 ml heparin-Sepharose (Amersham Biosciences) column equilibrated to the same conditions. The column was subjected to a linear gradient of 100 mM-1M NaCl in 20 mM Tris-HCl and the eluate collected in fractions. Fractions were analyzed by 8% SDS-PAGE and those containing Pfu-Pol were pooled and concentrated using Vivaspin 50000Da molecular weight cut off spin concentrators (Vivascience). Proteins were stored in 50 mM Tris-HCl (pH 8.0), 0.1 mM EDTA, 1 mM DTT, 0.1% NP40, 0.1% Tween 20, and 50% Glycerol.

A Single Base Primer Extension Assay

Referring to FIG. 3, there is shown a single base primer extension assay using the variant polymerises in accordance with the invention.

Method

Direct incorporation of correct and incorrect dNTPs was measured under the following conditions. 1.5 μM Pfu polymerase, 10 nM primer-template duplex (primer sequence, 5′-GGCGCCCGCGG-3′ (SEQ ID NO. 16); template sequence, 5′-GAAGCTCCGCGGGCGG-3′ (SEQ ID NO. 17)), 100 μM dATP or dGTP, 20 mM Tris-HCl (pH 8.0), 10 mM KCl, 10 mM (NH₄)₂SO₄, 0.1% Triton X-100®, 12.5 mM MgCl₂. The primer oligonucleotides were radiolabelled at their 5′-ends with ³²P[phosphate]. ³²P[phosphate] reactions were conducted at 50° C. and initiated by addition of MgCl₂. Samples were taken at 7, 14, 25, 35, 60 and 300 seconds. Reactions were terminated by addition of EDTA to a final concentration of 25 mM, analyzed on 20% denaturing polyacrylamide gels. Data was visualized using phosphoroimaging with Fuji BAS-MP image intensifying screens and quantified using the accompanying software package TINA version 2.9.

Discussion

The first free base in the template is T and the second dC. A normal polymerase (of expected high fidelity) should, therefore, extend the primer first by incorporation of dA (from dATP; 1^{st} base addition) and secondly by incorporation of dG (from dGTP; 2^{nd} base addition). This behaviour is seen by the wild type enzyme as shown in FIG. 3. When only dATP is supplied, first base addition is very rapid (corresponding to placing dA opposite T) but little addition of a second base (which would correspond to placing dA opposite dC) is observed. Correspondingly when only dGTP is supplied hardly any addition of the first base (which would correspond to dG addition opposite T) is seen.

The behaviour of low fidelity mutants is exemplified by D473G as shown in FIG. 3. Supplying dATP leads to rapid incorporation of the first base (corresponding to dA addition opposite T) but reasonably rapid addition of a second base (corresponding to aberrant addition of dA opposite dC) is also seen. With dGTP reasonably rapid addition of a first base (corresponding to aberrant addition of dG opposite T) is seen.

From the results shown in FIG. 3, the inventors noted that the polymerases having a mutated amino acid residue 471 (T471G and T471A), showed a fidelity similar to that of the wild-type. However a more sensitive assay, which is described at the end of this Example, which is based on amplification of the lacIOZα sequence (Cline, J., Braman, J. C., and Hogrefe, H. H. 1996 Nucleic Acids Research 24, 3546-3551) demonstrated that T471G and T471A were, respectively 1.2 and 1.6-fold less accurate than the D215A variant from which they were derived. Such small but valuable changes could not be appreciated in the experiments described in this Example.

As shown in FIG. 3, mutations to D473 (D473G and D473A) show reduced fidelity as compared to wild type. Mutations to Q472 (Q472G, Q472A and Q472P) also show reduced fidelity as compared to wild type, although the effect is not as severe as mutations to D473.

Mutation Spectrum produced by Pfa-Pol(D215A, D473G)

Referring to FIG. 4, there is shown a Mutation spectrum produced by Pfa-Pol(D215A, D473G).

The Pfa-Pol double mutant (D215A,D473G) was used in a PCR reaction to amplify the entire plasmid pET17b(Pfu-Pol)—this comprises the standard pET17b plasmid with a gene coding for wild type Pfu-Pol inserted at the multiple cloning site.

The following conditions were used: 2.5 units of polymerase, 20 mM Tris-HCl (pH between 8.0 and 8.8), 2 mM MgSO₄, 10 mM KCl, 10 mM (NH₄)₂SO₄, 0.1% Triton X-100®, 0.1 mg/ml BSA, 55 ng of pET17b(Pfu-Pol), 125 ng each primer and between 200-500 μM each dNTP. Reactions where subjected to 1 cycle of 95° C. for 30 seconds to denature all double stranded DNA, followed by 25 cycles of 95° C. for 30 seconds to separate plasmid strands, 55° C. for 30 seconds to anneal the primers, 68° C. for 15 minutes to extend the primers.

The amplified plasmid sequences obtained (67796 ng) were incubated with 10 units of DpnI restriction endonuclease at 37° C. for 2 hours. 1 μl of the DpnI digested products were used to transform E. coli XL-10 Gold (Stratagene) to ampicillin resistance (as directed in the Stratagene catalogue). Plasmids were isolated from the resultant colonies by miniprep (Qiagen) and sequenced (Lark Technologies Inc). Plasmids resulting from the PCR amplification were distinguished from starting plasmid by the presence of a unique codon alteration, introduced by the primers used for PCR. Plasmids resulting from PCR amplification were scored for mutations by comparing the first 500 bp of sequence (starting at the gene coding for Pfu-Pol) to that of the input wild type pET17b(Pfu-Pol) sequence. Nineteen plasmids were sequenced corresponding to a total number of bases of 9,500.

Discussion

In the 9,500 base-pairs sequenced, the inventors found a total of 68 mutations which are shown in the table of FIG. 4, and also represented by the bar chart. A-G means that where a dA base should have been incorporated opposite a T, a dG has been found instead. The same applies for the other base substitutions. Insertions and deletions mean that a base has been inserted or deleted. Just considering the base substitutions, there are eight possibilities. Therefore, if the mutation spectrum produced by Pfu-Pol(D215A,D473G) is completely random, each should occur with an equal probability of 8.3% (shown by the dotted line on the bar chart). While the spectrum is not completely unbiased (in particular T-A is overrepresented and G-A under represented), the degree of bias is low and the best observed so far.

Referring to FIG. 5, there is represented the degree of bias indication using the polymerase. The goal of error prone PCR of a DNA sequence is to change the encoded protein sequence in as random a manner as possible. Alteration of a base will change the amino acid incorporated into the protein (ignoring the redundancy within the genetic code). Therefore the most logical assessment of bias in a mutagenic polymerase is its propensity to alter any given base to any other base. Ignoring insertions and deletions, an unbiased polymerase would exhibit a base mutation profile of:—

(A→(G,C,T)=T→(G,C,A)=C→(T,A,G)=G→(C,T,A)=25%

(A→(G,C,T) means changing A to G or C or T). The same applies to the other bases.

Using this metric the bias indication figure for Pfu-Pol(D215A,D473G) shows a low level of bias, indicative of effective random mutagenesis. The mutation rates of each of the four bases base are very similar and near the expected figure of 25%.

Furthermore, from a knowledge of the total of 68 mutations found in the 9,500 base-pairs sequenced, together with the known amount of input DNA (55 ng) and product DNA (67797 ng) it is possible to calculate the error rate of the polymerase from the following equation (Cline, J., Braman, J. C., and Hogrefe, H. H. 1996 Nucleic Acids Research 24, 3546-3551):—
Error Rate=Mutation frequency/number of doublings(d)
Where

d=log₁₀(product DNA/input DNA)/log₁₀2

d=log₁₀(67797/55)/log₁₀2

d=10.2
Error Rate=(68/9,500)/10.2=7×10⁻⁴

Under identical conditions Pfu-Pol (D215A) from which the D215A,D473G double mutant is derived has an error rate of 5.8×10⁻⁵. Therefore, the D473 mutation decreases fidelity by a factor of 12.

1. A variant archaeal DNA polymerase comprising a modified amino acid sequence of a wild-type amino acid sequence, wherein during DNA replication, the variant polymerase is adapted to mis-incorporate a greater number of incorrect bases than compared to a wild-type archaeal DNA polymerase.

2. The variant archaeal DNA polymerase according to claim 1, wherein the variant is derived from any one of the archaeal family DNA polymerases illustrated in FIG. 1.

3. The variant archaeal DNA polymerase according to claim 1, wherein the variant is derived from an archaeal family B DNA polymerase.

4. The variant archaeal DNA polymerase according to claim 1, wherein the variant polymerase is derived from a family independently selected from a group consisting of: Eutyarchaeota; Crenarclzaeota; and Nanoarchaeota.

5. The variant archaeal DNA polymerase according to claim 1, wherein the variant polymerase is derived from a genus independently selected from a group consisting of: Thermococci; Methanococci; Thermoplasmata; Methanobacteria; Archaeoglobi; Methanopyrii; Thermoprotei; and Nanoarchaeum.

6. The variant archaeal DNA polymerase according to claim 1, wherein the variant polymerase is derived from a species independently selected from a group consisting of: Pyrococcus furiosus (NP577941); Pyrococcus woesei (P61876); Pyrococcus ST700 (CAC12847); Thermococcus 9°N-7 (1QHTA); Thermococcus gorgonarius (P56689); Thermococcus fumicolans (AA93738); Pyrococcus horikoshii (C71210); Pyrococcus abysii (CAA90888); Pyrococcus GE23 (CAA90887); Pyrococcus kodakaraensis (KOD1)(1GCXA); Thermococcus GE8 (CAC12850); Thermococcus hydrothermalis (CAC 18555); Pyrococcus GB-D (deep vent)(AAA67131); Pyrococcus glycovorans (CAB81809); Thermococcus litoralis (vent) (P30317); and Thermococcus TY (CAA73475).

7. The variant archaeal DNA polymerase according to claim 1 having a modified amino acid sequence of wildtype Pyrococcus furiosus DNA polymerase (Pfu-Pol).

8. The variant archaeal DNA polymerase according to claim 1 having at least one modification in amino acid residues equivalent to the residues forming a loop (or hinge region) between two alpha helices, known as N alpha helix and 0 alpha helix of Pyrococcus furiosus DNA polymerase.

9. The variant archaeal DNA polymerase according to claim 1, wherein the variant comprises at least one modification in amino acids equivalent to 471, 472, and/or 473 of Pyrococcus furiosus DNA polymerase.

10. The variant archaeal DNA polymerase according to claim 9, wherein the modification at amino residue 471, 472 or 473 comprises a substitution with a small or constraining amino acid residue.

11. The variant archaeal DNA polymerase according to claim 10, wherein the amino acid may include glycine, alanine, proline, or serine.

12. The variant archaeal DNA polymerase according to claim 1 with a mutation at amino acid 471.

13. The variant archaeal DNA polymerase according to claim 12, wherein the mutation is T471G or T471A.

14. The variant archaeal DNA polymerase according to claim 1 with a mutation at amino acid 472.

15. The variant archaeal DNA polymerase according to claim 14, wherein the mutation is Q472G, Q472A or Q472P.

16. The variant archaeal DNA polymerase according to claim 1 with a mutation at amino acid 473.

17. The variant archaeal DNA polymerase according to claim 16 wherein the modification is D473G or D473A.

18. The variant archaeal DNA polymerase according to claim 1 with a mutation at amino acid 215.

19. The variant archaeal DNA polymerase according to claim 18, wherein the modification is D215A.

20. A nucleic acid molecule encoding an archaeal DNA polymerase according to claim 1.

21. A method of using a variant archaeal DNA polymerase according to claim 1, in random mutagenesis protocols.

22. A method of causing random mutations in DNA comprising the steps of:
(i) denaturing a double strand of DNA by heating a solution containing the DNA, free oligonucleotides, primers and a variant archaeal DNA polymerase according to claim 1;
(ii) reducing the temperature of the solution to effect annealing of the primer and the DNA; and
(iii) allowing extension of the DNA strand by the variant polymerase.

23. A kit for random mutagenesis protocols comprising a variant archaeal DNA polymerase according to claim 1; and optionally buffers and free bases.

24. A variant DNA polymerase comprising a modified amino acid sequence of a wild-type amino acid sequence, the variant comprising at least one modification in amino acids equivalent to residues 471, 472, or 473 of Pyrococcus furiosus DNA polymerase, wherein during DNA replication, the variant polymerase is adapted to mis-incorporate a greater number of incorrect bases than compared to a wild-type DNA polymerase.

25. The variant DNA polymerase according to claim 24, wherein the DNA polymerase is a DNA polymerase delta (S).

26. The variant DNA polymerase according to claim 24, wherein the variant DNA polymerase is derived from a eukaryote.

27. The variant DNA polymerase according to claim 26, wherein the variant DNA polymerase is derived from a mammal or fungus.

28. The variant DNA polymerase according to claim 25, wherein the variant DNA polymerase is derived from a eukaryote.
